(19) United States
(12) Patent Application Publication
(10) Pub. No.: US 2025/0163089 A1
(43) Pub. Date: May 22, 2025

(54) Branched Molecules for Redox Flow Batteries

(71) Applicant: XL Batteries, Inc., Marlborough, MA (US)

(72) Inventors: Paul Evans, Marlborough, MA (US); Thomas Sisto, Marlborough, MA (US); Anthony Bencivenga, Marlborough, MA (US); Anouck Champsaur, Marlborough, MA (US)

(73) Assignee: XL Batteries, Inc., Marlborough, MA (US)

(21) Appl. No.: 18/951,491

(22) Filed: Nov. 18, 2024

Related U.S. Application Data
(60) Provisional application No. 63/600,534, filed on Nov. 17, 2023.

(51) Int. Cl. C07F 17/02; H01M 8/18

(52) U.S. Cl. CPC C07F 17/02; H01M 8/188

(57) ABSTRACT

Compounds with redox molecular cores and branched molecular tails comprising ionic functionalities for uses as electrolytes in redox flow batteries are described, as well as methods of synthesis and use thereof. The compounds have redox active perylene diimide or ferrocene molecular core decorated with the branched molecular tails having multiple ionic functionalities to improve water solubility of the molecular core, and, as such, its performance as the electrolyte.

### CROSS-REFERENCE TO RELATED APPLICATIONS

The present application claims priority to U.S. Provisional Patent Application No. 63/600,534 entitled “Branched Molecules for Redox Flow Batteries” to Evans, filed Nov. 17, 2023, the disclosure of which is incorporated herein by reference in their entireties.

### FIELD OF THE INVENTION

The invention is generally directed to redox active compounds with solubilizing branched molecular tails bearing ionic salt functionalities suitable for use as electrolytes in redox flow batteries or other charge storage applications.

### BACKGROUND OF THE INVENTION

A Redox Flow Battery (RFB) is a battery in which chemical energy is provided by circulating two electrolyte solutions with chemically distinct compositions through adjacent compartments of the battery separated by a membrane, such that ion transfer inside the battery (accompanied by a current flow through an external circuit) occurs across the membrane, while the electrolyte solutions circulate in their respective spaces. Accordingly, electrolyte solution parameters such as electrolyte's solubility, viscosity, and conductivity are key to redox flow battery's viability and performance. Still, safety, long operation life, and the design principle that decouples energy and power make RFB a promising technology for large-scale energy storage. However, redox flow batteries often suffer from problems associated with high cost and low energy density, which can limit their applications. Accordingly, there exists a need for low cost RFB designs and components that can improve RFB performance.

### SUMMARY OF THE INVENTION

Various embodiments are directed to redox active compounds with solubilizing branched molecular tails bearing ionic salt functionalities suitable for use as electrolytes in redox flow batteries or other charge storage applications.

Various such embodiments are directed to compounds for use as an electrolyte in a redox flow battery including:
a molecular core, wherein the molecular core is a redox moiety, covalently functionalized with
one or more branched molecular tails, wherein the one or more branched molecular tails, together, further comprise
at least two ionic functionalities.

In still various embodiments, the molecular core is perylene diimide, or ferrocene.

In yet various embodiments, the molecular core is perylene diimide and the compound has a Formula (I):

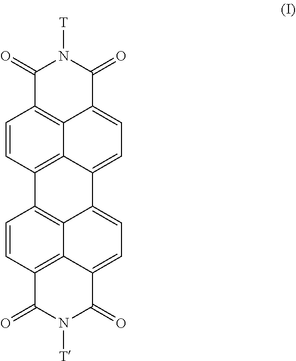

or a salt thereof, wherein:
T and T′ are, independently selected from the group consisting of:

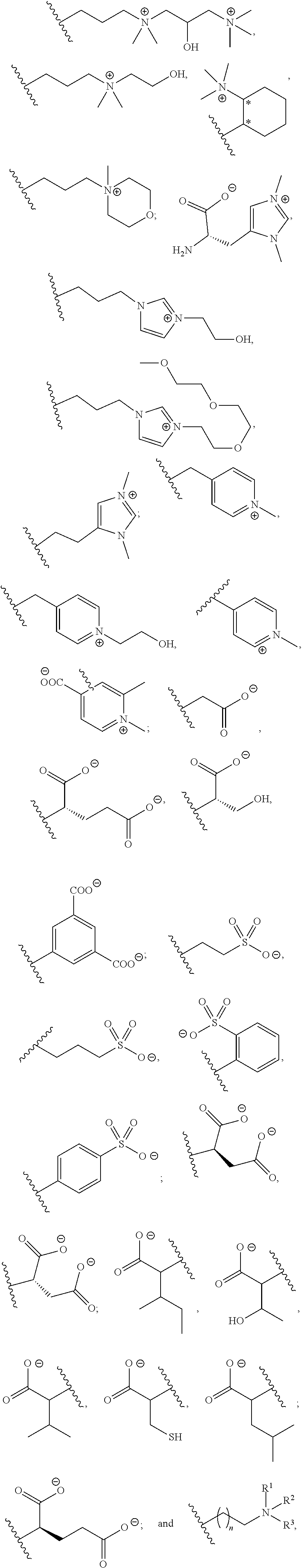

wherein R¹, R², R³ is, independently, an alkyl, a glycol oligomer, or an alkyl alcohol.

In still yet various embodiments, the molecular core is perylene diimide and the compound has a Formula (I):

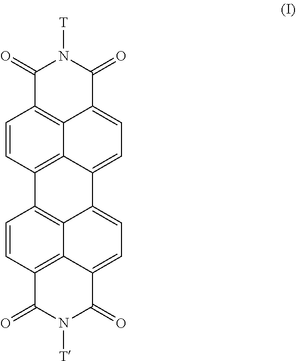

or a salt thereof, wherein:
T is -(L-G)ₙ-X;
T′ is —H, —(C₁-C₆)alkyl, or -(L-G)ₙ-X;
L is —(C₂-C₅)alkyl, optionally substituted with —OH, —OCH₃, -halogen;
G is a moiety selected from the group consisting of:

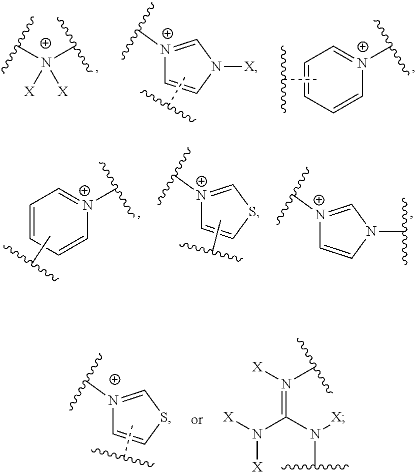

each X is, independently, —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups;
each R¹ is independently —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂;
n=2 to 8; and
p=3 to 20.

In yet still various embodiments, T and T′ are each, independently, -(L-G)ₙ-X.

In still yet various embodiments, T and T′ are the same.

In yet still various embodiments, T and T′ are different.

In still yet various embodiments, L is unsubstituted —(C₂-C₅)alkyl.

In yet still various embodiments, L is ethyl or propyl.

In still yet various embodiments, n is 2, 3, or 4.

In yet still various embodiments, G is

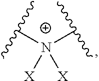

In still yet various embodiments, X is —H, -methyl or —CH₂CH₂OH.

In yet still various embodiments, each X is, independently, —H or —(C₁-C₆)alkyl.

In still yet various embodiments, at least one X is —CH₃CH₂OH.

In yet still various embodiments, the compound of Formula (I) is a compound selected from the group consisting of:

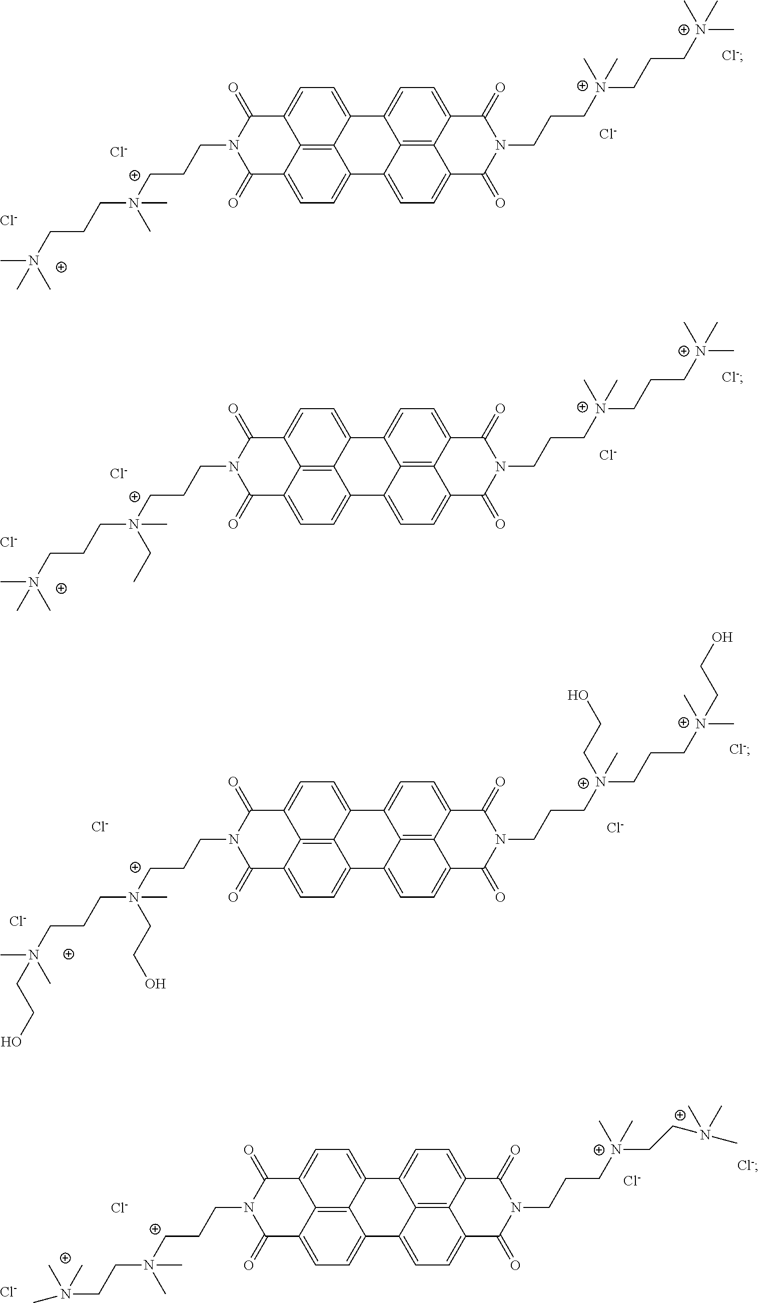

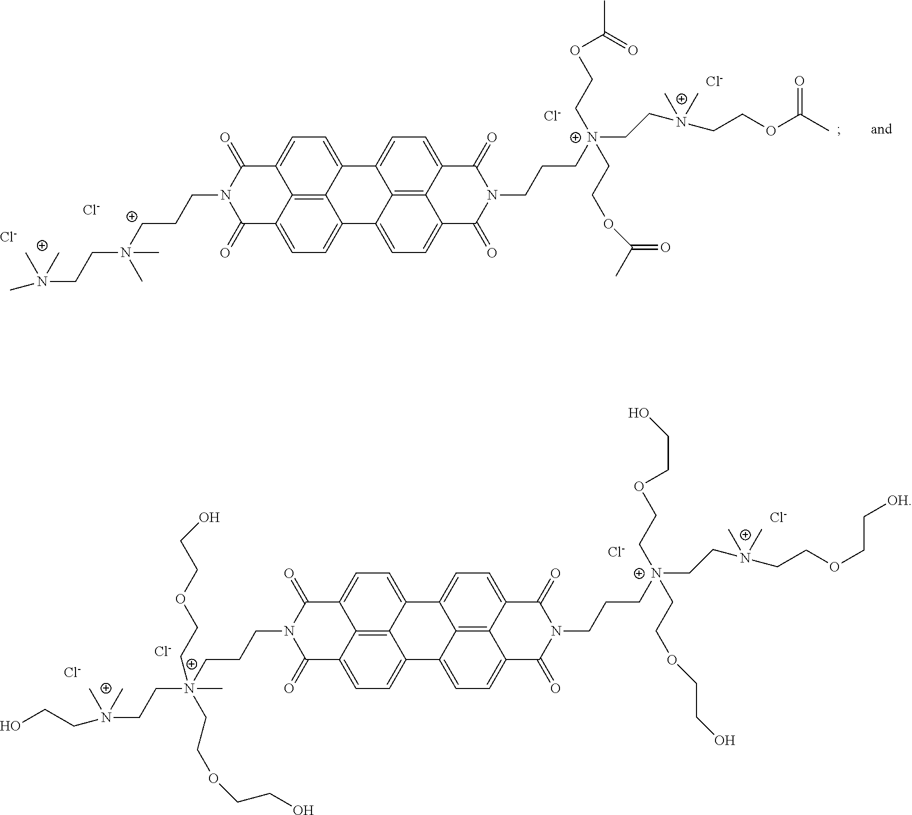

In yet still various embodiments, the molecular core is perylene diimide and the compound has a Formula (II):

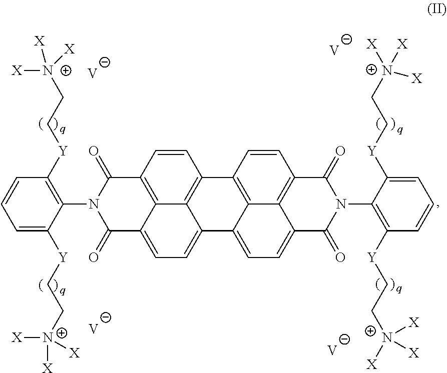

wherein
each Y is, independently, —O—, —S— or —NH—;
each q is, independently, 1 to 8; and
each X is, independently, selected from the group consisting of: —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups;
each R¹ is, independently, selected from the group consisting of: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂; and
each V is a counterion.

The compound of claim 16, wherein the compound of Formula (II) is:

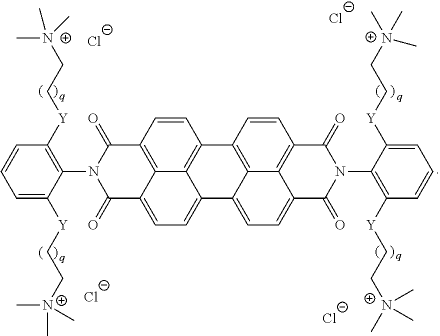

In yet still various embodiments, the molecular core is perylene diimide and the compound has a Formula (III):

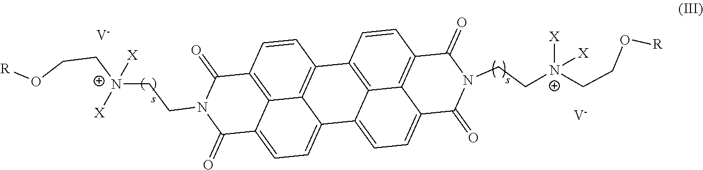

wherein:
each X is, independently, selected from the group consisting of: —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups;
each R¹ is, independently, selected from the group consisting of: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆)alkyl, —O(C═O)(C₁-C₆)alkyl, —O(C═O)O(C₁-C₆)alkyl, O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂;
each s is, independently, 2 to 4;
each R is, independently, —H, —CH₂OH, —CH₂CH₂OH, —CH₂CH₂OCH₂CH₂OH, or —CH₂CH₂OCH₂CH₂O(C₁-6)alkyl; and
each V⁻ is a counterion.

In yet still various embodiments, the compound of Formula (III) is:

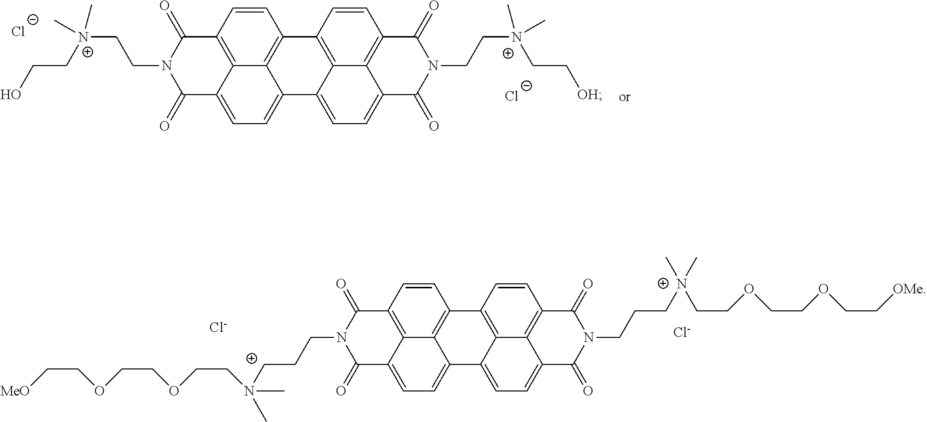

In still yet various embodiments, the molecular core is perylene diimine and the compound has a Formula (IV):

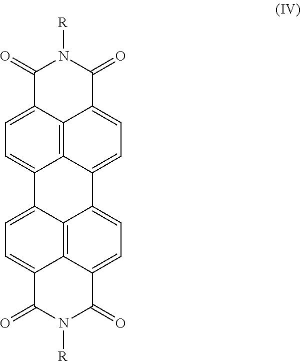

wherein
R is

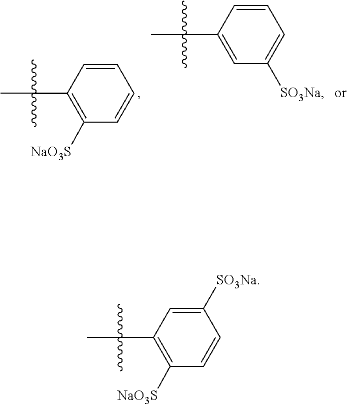

In yet still various embodiments, the compound of Formula (IV) is:

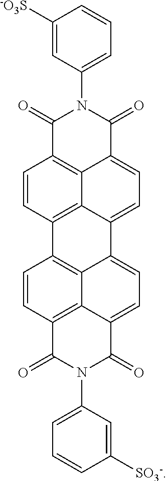

In still yet various embodiments, the molecular core is perylene diimide and the compound has a Formula (V):

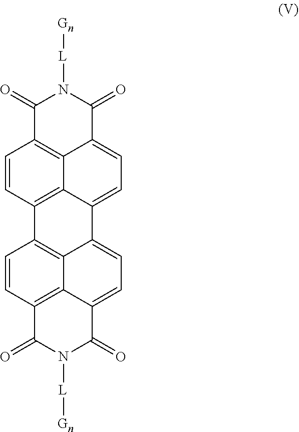

or a salt thereof, wherein
L is —(C₁-C₆)alkyl;
each G is

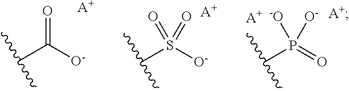

A⁺ is one or more cations; and
n=1 to 5.

In yet still various embodiments, L is substituted with —OH, —OCH₃, or -halogen.

In still yet various embodiments, each A⁺ is one or more cations selected from the group consisting of: Li⁺, K⁺, Na⁺, NH₄⁺, and another suitable cation.

In yet still various embodiments, each G is

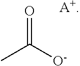

In still yet various embodiments, each L is propyl.

In yet still various embodiments, n is 2.

In still yet various embodiments, L-Gₙ of Formula (V) has at least one chiral center.

In yet still various embodiments, the formula (V) has at least one stereoisomer.

In still yet various embodiments, the compound of Formula (V) is:

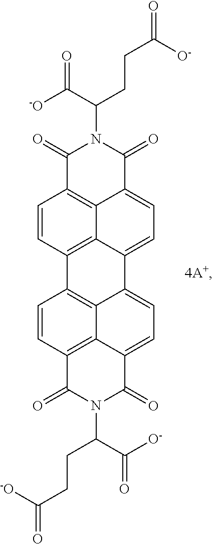

and any one or any combination of its stereoisomers:

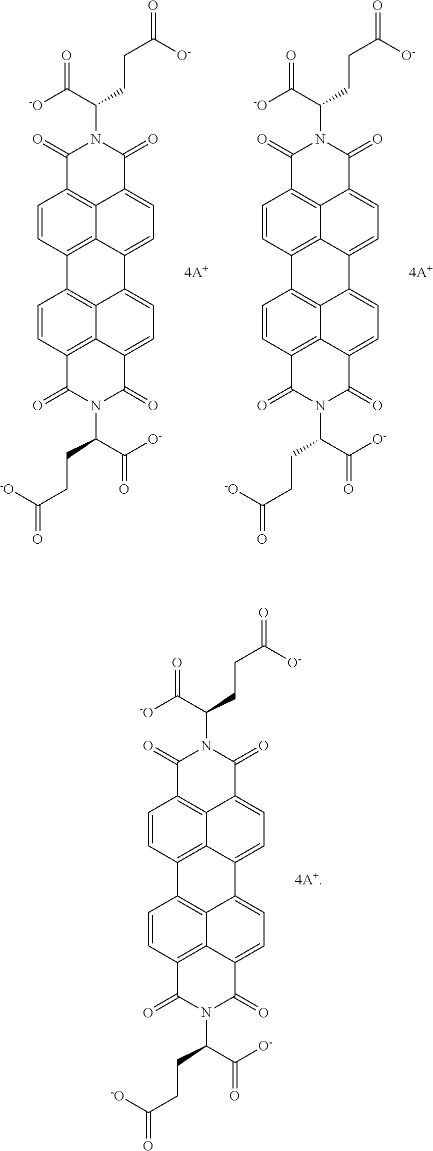

In yet still various embodiments, A⁺ is one or more cations selected from the group consisting of: Li⁺, K⁺, Na⁺, NH₄⁺, and another applicable.

In still yet various embodiments, the compound of Formula (V) is:

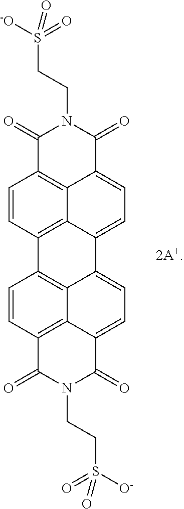

In yet still various embodiments, the molecular core is ferrocene and the compound has a structure selected from the group consisting of:

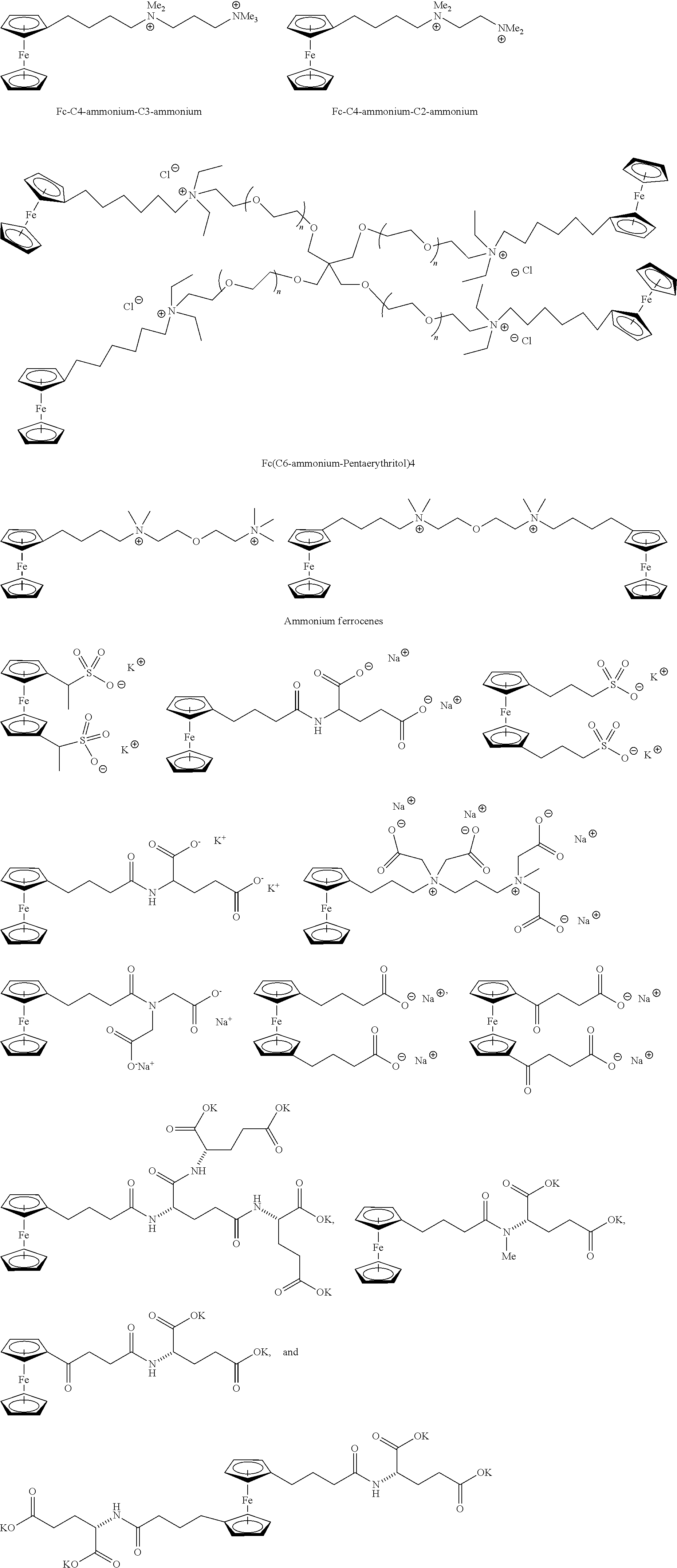

In still yet various embodiments, the molecular core is ferrocene and the compound has a Formula VI:

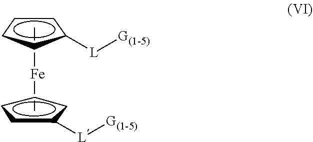

wherein:
L_{F} is selected from the group consisting of: —(C₁-C₁₀)alkyl, —(C₁-C₅)alkenyl, —(C1-C6)alkynyl, —(C₁-C₆)alky-O(C₁-C₆)alkyl, —(C₁-C₆)alkyl-O—(C═O)—(C₁-C₅)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
L_{F}′ is selected from the group consisting of: —H, —(C₁-C₁₀)alkyl, —(C₁-C₆)alkenyl, —(C1-C6)alkynyl, —(C₁-C₆)alkyl-O(C₁-C₆)alkyl, —(C₁-C₆)alkyl-O—(C═O)—(C₁-C₆)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
G^{F} is selected from the group consisting of:

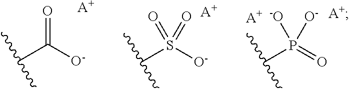

G^{F} is greater than or equal to 2;
A⁺ is one or more cations and
R² is —(C₁-C₁₀)alkyl, —(C₁-C₆)alkenyl, —(C₁-C₆)alkynyl, —(C₁-C₁₀)alkyl-aryl, -aryl, or —(C═O)—(C₁-C₆)alkyl.

In yet still various embodiments, L^{F} is substituted by at least one group selected from the group consisting of: G^{F}, —OH, —OCH₃, and -halogen.

In still yet various embodiments, L_{F}′ is substituted at least one group selected from the group consisting of: G^{F}, —OH, —OCH₃, and -halogen.

In yet still various embodiments, R² is substituted by at least one G^{F}.

In still yet various embodiments, A⁺ is one or more cations selected from the group consisting of: Li⁺, K⁺, Na⁺, NH₄⁺, and another suitable cation.

In yet still various embodiments, L_{F}, L_{F}′, and or G^{F}ₙ has at least one chiral center.

In still yet various embodiments, the compound has at least one stereoisomer.

In yet still various embodiments, the compound of Formula (VI) is

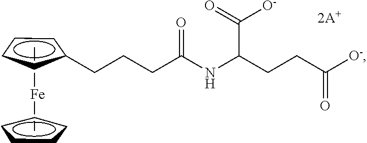

and any one or any combination of its stereoisomers:

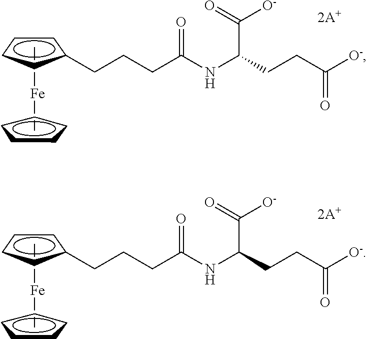

The compound of claim 34, wherein the compound of Formula (VI) is:

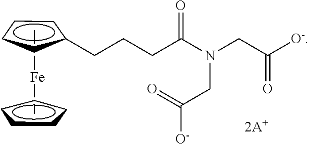

Many embodiments are directed to redox flow batteries including:
a first half-cell comprising a first electrode and a first solution of a first electrolyte, wherein the first electrolyte is the compound with the perylene diimide molecular core as set forth in the various embodiments above;
a second half-cell comprising a second electrode and a second solution of a second electrolyte; and
a separator interposed between the first half-cell and the second half cell.

In still many such embodiments, the second electrolyte is the compound with the ferrocene molecular core of any one of various embodiments above.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which form a part of this disclosure.

### BRIEF DESCRIPTION OF THE DRAWINGS

These and other features and advantages of the present invention will be better understood by reference to the following detailed description when considered in conjunction with the accompanying data and figures, wherein:

FIG. 1 schematically illustrates a perylene diimide molecular core, and the formation of π-π stacks it undergoes upon dissolution in water, according to prior art.

FIGS. 2A and 2B provide illustrative examples of compounds that can be used to modify the perylene diimide molecular core with solubilizing branched molecular tails, in accordance with embodiments of the application.

FIG. 3 schematically illustrates the compound comprising the perylene diimide molecular core (101) enhanced with two branched molecular tails (104), wherein each branched molecular tail, in turn, comprises two ionic functionalities (105) repulsed by electrostatic repulsion forces (106), and such compound's dissolution behavior in water, in accordance with embodiments of the application.

### DETAILED DISCLOSURE

Turning now to the schemes, images, and data, embodiments of a compound for use as an electrolyte in a redox flow battery (RFB) are described, as well as methods of synthesis, and use thereof in RFBs or other charge/energy storage applications. In many embodiments, the compound is a redox compound. Many such embodiments are directed to a compound comprising a perylene diimide (PDI) core further comprising one or more branched molecular tails, wherein the branched molecular tails combined comprise two or more ionic functionalities. However, in some embodiments, the compound comprises a ferrocene (Fc) core decorated by the one or more molecular tails with the two or more ionic functionalities overall. In many embodiments, the compound is characterized by an improved solubility in aqueous and or organic solvents, as compared to analogs without the branched molecular tails, or with comparable linear molecular tails, or with branched molecular tails lacking ionic functionalities. In many embodiments, an electrolyte solution comprising the compound is characterized by a lower viscosity and a higher conductivity, as compared to an analogous electrolyte solution comprising an analog of the compound without the branched molecular tails, or with solubilizing molecular tails that are comparable to the compound's in size and chemical composition but lack either branching, or ionic functionalities, or both. In many embodiments, the compound is water soluble. In many embodiments, an aqueous solution comprising the compound, i.e., an aqueous electrolyte solution, is characterized by a neutral pH.

Furthermore, certain embodiments are directed to a redox flow battery comprising the aqueous electrolyte solution comprising the compound as the electrolyte. In many such embodiments, the redox flow battery is characterized by enhanced stability, wherein the redox flow battery maintains its performance under working conditions for weeks, months, or longer. In many embodiments, the redox flow battery comprising the compound as its electrolyte component is characterized by a higher energy density as compared to conventional RFBs.

It will be understood that the embodiments of the invention described herein are not intended to be exhaustive or to limit the invention to precise forms disclosed. Rather, the embodiments selected for description have been chosen to enable one skilled in the art to practice the invention.

Here, the terms “branching,” “branched,” “branched molecules,” “branched chains,” “branched side chains,” and “branched molecular tails” are used interchangeably, and refer to a regular or irregular covalent attachment of side chains to a backbone chain of at least one monomer of a polymer. Furthermore, branching can occur by the replacement of a substituent (e.g. a hydrogen atom) on a monomer subunit by another covalently-bonded chain of that polymer; or by a chain of another type. A “branch” may comprise one atom extending from another (“main”) chain of at least three atoms.

Here, “ionic groups” or “charged groups” refer to chemical groups having ions that form charged particles when an atom (or a group of atoms) gains or loses electrons. A cation is a positively charged ion, and an anion is a negatively charged ion. “lonic” and “charged” are used interchangeably. Furthermore, here, “ionic chains,” “charged chains,” “ionic side chains,” “charged side chains,” “ionic tails,” and “charged tails” all refer to branched chains comprising ionic groups, unless specified otherwise. The ionic chains can be positively charged or negatively charged.

Electrolyte solutions for use in Redox Flow Batteries (RFBs) must possess a number of advantageous characteristics in order to enable viable battery performance. These characteristics include low viscosity, high conductivity, and high solubility of utilized electrolytes. It should be noted here that, although not to be bound by any theory, an electrolyte solution conductivity is typically impacted by the solution's viscosity, wherein lower viscosity affords higher conductivity. In addition, low cost and tunable structure are also highly desirable characteristics for the organic redox-active materials for applications as organic RFB electrolytes, wherein tuning the molecular structure of the redox molecule can change its solubility, which, in turn, contributes directly to the energy density in the organic redox flow battery relying on such redox molecule.

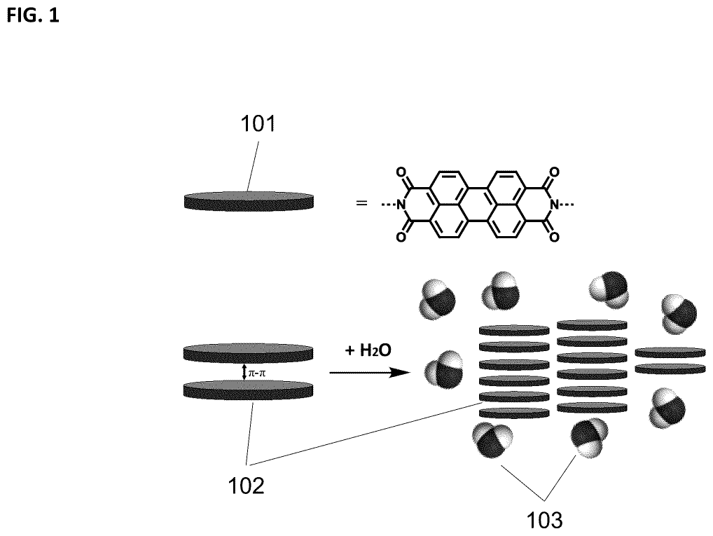

Perylene diimide (PDI), a compound with a molecular core illustrated by moiety 101 in FIG. 1, possesses advantageous redox properties and capabilities, which, in principle, make it suitable for application as an electrolyte in RFBs. More specifically, the highly-conjugated, electron-poor PDI is readily amenable to reversible reduction to accept two electrons. However, PDI is characterized by poor water solubility, due to its tendency towards π-π stacking interactions between the aromatic core units in water, leading to formation of aggregates 102, as schematically shown in FIG. 1. More specifically, as shown in FIG. 1, the π-π stacking aggregation of perylene diimide molecular cores excludes water molecules (103) and, thus, inhibits dissolution of PDI by water.

Conventionally, water solubility of perylene diimide is aided by introducing solubilizing N-substituents (i.e., imide-position substituents), such as, for example, hydrophilic molecular tails/chains, including, as a more specific example, glycol chains. However, additional new approaches to solubilizing PDI-based electrolytes are highly sought after in order to further improve this electrolyte's solubility in water, as well as viscosity and conductivity of PDI-containing electrolyte solutions and, as such, advance the field of RFBs.

Accordingly, this application is directed to embodiments of a compound comprising a perylene diimide (PDI) molecular core further comprising one or more branched molecular tails, wherein the totality of the branched molecular tails comprises two or more persistent ionic functionalities/salts. However, in some embodiments, the compound comprises a ferrocene (Fc) molecular core decorated by the one or more molecular tails comprising the two or more persistent ionic salts. In some embodiments, each individual branched molecular tail comprises zero, one, two, or more ionic functionalities, however, the total number of the ionic functionalities for all the branched molecular tails of the compound exceeds one. In other words, in many embodiments, the number of the ionic functionalities in the compound is two or more, regardless of the number of the branched molecular tails in the compound. In many such embodiments, the compound is characterized by an improved solubility in aqueous and or organic solvents, as compared to analogs without the branched molecular tails, or analogs with linear molecular tails of comparable size, or analogs with comparable branched molecular tails without ionic salt functionalities. In many embodiments, an electrolyte solution comprising the compound is characterized by a lower viscosity and a higher conductivity, as compared to an analogous electrolyte solution comprising an analog of the compound without the branched molecular tails, or with solubilizing molecular tails that are comparable to the compound's in size and chemical composition but lack either branching, or ionic functionalities, or both. More specifically, in many embodiments, the compound is water soluble, and an aqueous solution comprising the compound, i.e., an aqueous electrolyte solution, is characterized by a neutral pH. In many embodiments, the compound is employed as an electrolyte in a redox flow battery, or in another charge storage device or application. In many such embodiments, the compound is used as an anolyte or catholyte. In many embodiments, the compound comprising the PDI molecular core is used an anolyte, while the compound comprising the Fc core is used as a catholyte, within the same redox flow battery, or independently.

PDI is a highly-conjugated, electron-poor molecule readily amenable to reversible reduction to accept two electrons. Accordingly, in many embodiments, wherein the compound comprises the PDI molecular core, the compound is employed as the anolyte of the redox flow battery or another charge storage device. In many such embodiments, the compound with the PDI molecular core, wherein the PDI molecular core is functionalized at one or both of its N atoms with at least one branched molecular tail, wherein all branched molecular tails, together, bear at least two ionic salt functionalities, is easily accessed via, for example, a synthetic route starting from perylene tetracarboxylic dianhydride and proceeding via condensation with primary amines-bearing branched molecular tails to covalently link the branched molecular tails to the PDI-molecular cores. In some other embodiments, the thus (or via another synthetic route) attached branched molecular tails are further synthetically modified post-attachment, including further extended and or further branched. In many embodiments, modifying the PDI molecular core of the compound with one or more branched molecular tails as described herein, favorably affects the solubility of the compound and, consequently, the viscosity and conductivity of the compound's solution, however these modifications do not significantly impact the charge storage stability of the perylene diimide molecular core. More specifically, it should be noted here that, although not being bound by any theory, electron and frontier molecular orbital densities of the perylene diimide core are concentrated in its aromatic backbone and, as such, are not affected by functionalization of PDI at its N-terminals. In other words, the redox properties of the PDI molecular core, including energy level, redox reversibility, and stability in solution, are unaffected by the N-functionalization of PDI, and, therefore, such functionalization affords N-functionalized perylene diimides with identical redox properties, regardless of the chemical identity and physical properties of the added functional groups (i.e., branched molecular tails with ionic salt components). Accordingly, in many embodiments, covalent modification of the PDI molecular core of the compound at one or both N-terminals does not influence the charge storage stability of the PDI molecular core.

Similarly, Fc is redox-active molecule that can be employed as a catholyte of the redox flow battery or another charge storage device. Accordingly, in many embodiments, wherein the compound comprises the Fc molecular core, the compound is employed as the catholyte of the redox flow battery or another charge storage device. In many embodiments, the compound with the Fc molecular core is employed as a catholyte in the redox flow battery. In many such embodiments, the compound comprises the Fc molecular core functionalized at one or both of its cyclopentadienyl rings with at least one branched molecular tail bearing, such that the totality of the branched molecular tails bears at least two ionic salt functionalities. In many embodiments, modifying the Fc molecular core of the compound with one or more branched molecular tails as described herein, favorably affects the solubility of the compound and, consequently, the viscosity and conductivity of the compound's solution.

Moreover, in many embodiments, the compound is highly stable both in charged and uncharged states. In many embodiments, wherein the compound comprises PDI molecular core, the compound shows good stability in the 2-electron reduced state, even when present in high concentrations in an aqueous media. Additionally, in many embodiments, the compound is compatible with both ion-exchange and size-exclusion membranes and demonstrates minimal crossover between the anode and cathode chambers of a battery.

In many embodiments, wherein the compound comprises the PDI molecular core, the one or more branched molecular tails with the persistent ionic functionalities are covalently attached to the compound via one or both imide nitrogens on the perylene diimide molecular core. In many other embodiments, wherein the compound comprises the Fc molecular core, the one or more branched molecular tails with the persistent ionic functionalities are covalently attached to the compound via one or both cyclopentadienyl rings of the ferrocene molecular core. In many embodiments, the one or more branched molecular tails comprise persistent ionic functionalities such that the total number of the persistent ionic functionalities per the compound is at least two. However, in some embodiments, each of the one or more branched molecular tails comprises at least two persistent ionic functionalities. In some embodiments, each of the one or more branched molecular tails comprises at least three persistent ionic functionalities. Yet, in some embodiments, each of the one or more branched molecular tails comprises at least four persistent ionic functionalities. Still, in some embodiments, each of the one or more branched molecular tails comprises at least five persistent ionic functionalities. In many embodiments, the persistent ionic functionalities of each branched molecular tail are the same functionality; while in many other embodiments, the persistent ionic functionalities of each branched molecular tail are different functionalities. In many embodiments, the persistent ionic functionalities are cationic. In some embodiments, the persistent ionic functionalities are anionic. In some embodiments, the persistent ionic functionalities are zwitterionic, and as such, the compound comprises both cationic and anionic ionic functionalities. In many embodiments, each of the persistent ionic functionalities is a functionality independently selected from the group comprising (but are not limited to): ammonium/ammonium salt, imidazolium/imidazolium salt, pyridinium/pyridinium salt, carboxylate/carboxylate salt, and sulfonate/sulfonate salt. In many embodiments, the ionic group as prepared/synthesized is neutral and requires activation, such as, for example, deprotonation or protonation, prior to use as the electrolyte. Accordingly, it should be noted here that, in many embodiments, wherein the redox flow battery employs as the electrolyte the compound modified with the branched molecular tails comprising the cationic functionalities, an anion-exchange membrane is used to separate the battery's anode and cathode compartments. However, in many embodiments, wherein the compound modified with the branched molecular tails comprising the anionic functionalities is used as the electrolyte, a cation exchange membrane is used to separate the battery's anode and cathode compartments.

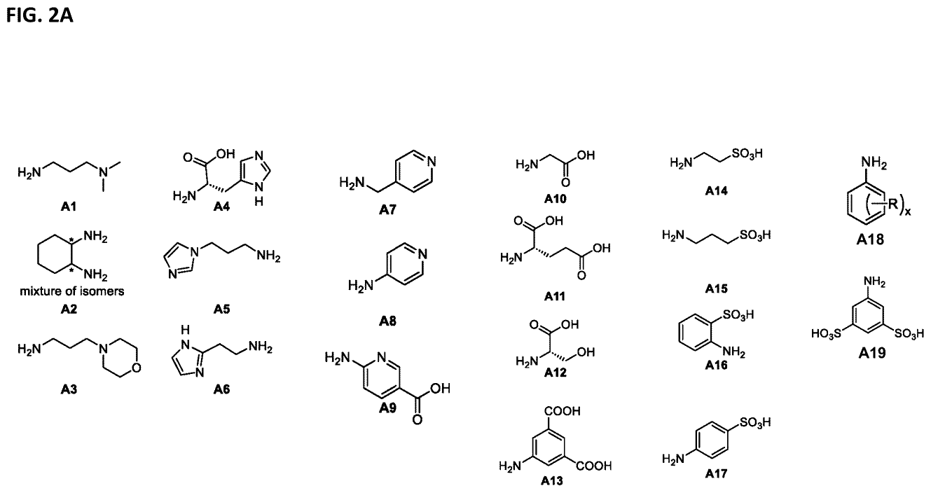

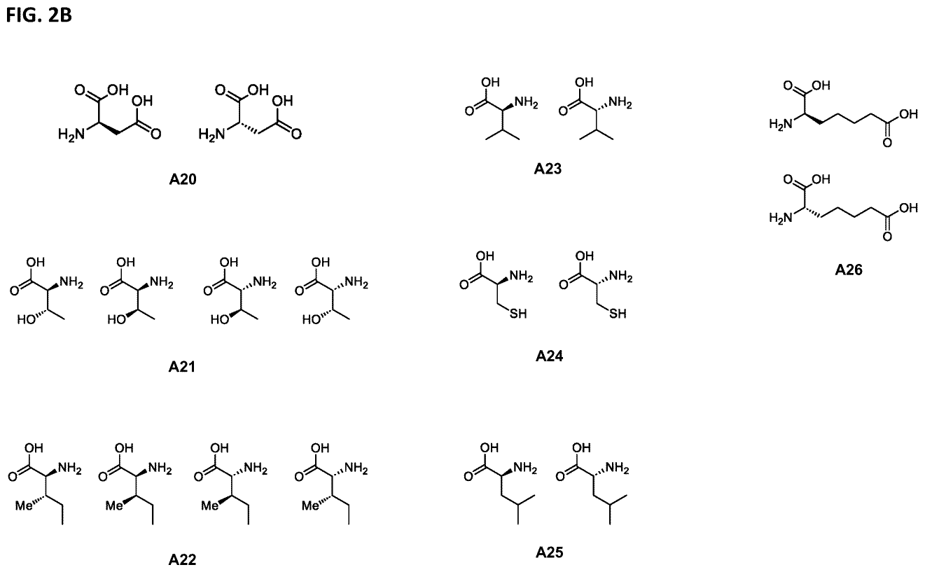

FIGS. 2A and 2B provide examples illustrating chemical structures of solubilizing branched molecular tails precursors that can be used to modify (e.g., directly add to the molecular core or further extend the branched molecular tails with) the molecular core of the compound as described herein according to many embodiments. More specifically, in many embodiments, the branched precursors to be used for modifying the molecular core of the compound, (or for further modifying the already attached branched molecular tails) are any one or a combination of compounds selected from the group comprising (but not limited to): ammonium compounds such as A1, A2, and A3; imidazolium compounds such as A4, A5, and A6; pyridinium compounds such as A7, A8, and A9; carboxylate compounds such as A10, A11, A12, and A13; sulfonate compounds such as A14, A15, A16, and A17; and compounds such as A18, wherein R is, independently, selected from the group of functionalities comprising: SO₃H, OH, NH₂, NMe₂, COOH, H, or any combination thereof, and n is an integer number of 1 to 5, including as A19. In many embodiments, the branched molecular tail is a moiety obtained from a chiral amine compound such as illustrated by compounds A20 through A26 in FIG. 2B. To this end, in many embodiments, wherein, for example, the branched molecular tail comprises pyridinium functionality as the ionic functionality, the compound comprising one or more branched molecular tails based on A9 is characterized by an improved water solubility and, thus, electrolyte solution conductivity, as compared to the compound comprising the same number of linear molecular tails based on unbranched A7 and or A8 moieties only. As another example, in many embodiments, wherein the branched molecular tail comprises carboxylate functionality, the compound comprising one or more branched molecular tails based on A11, A12, and A13 is characterized by an improved water solubility and, thus, electrolyte solution conductivity, as compared to the compound comprising the same number of linear molecular tails based on unbranched A10 moiety only. In many embodiments, as yet another example, wherein the branched molecular tail comprises sulfonate functionality, the compound comprising one or more branched molecular tails based on A16 and A17 is characterized by an improved water solubility and, thus, electrolyte solution conductivity, as compared to the compound comprising the same number of linear molecular tails based on unbranched A14 and A15 moieties only.

To this end, in many embodiments, wherein the molecular core is PDI, the compound has the structure of Formula (I):

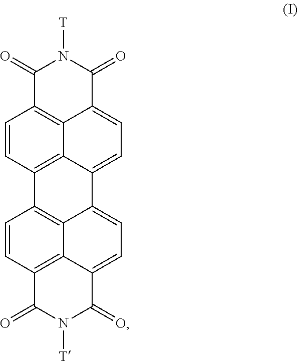

or a salt thereof, wherein T and T′, i.e., the branched molecular tails, each, independently is a moiety selected from the group comprising:

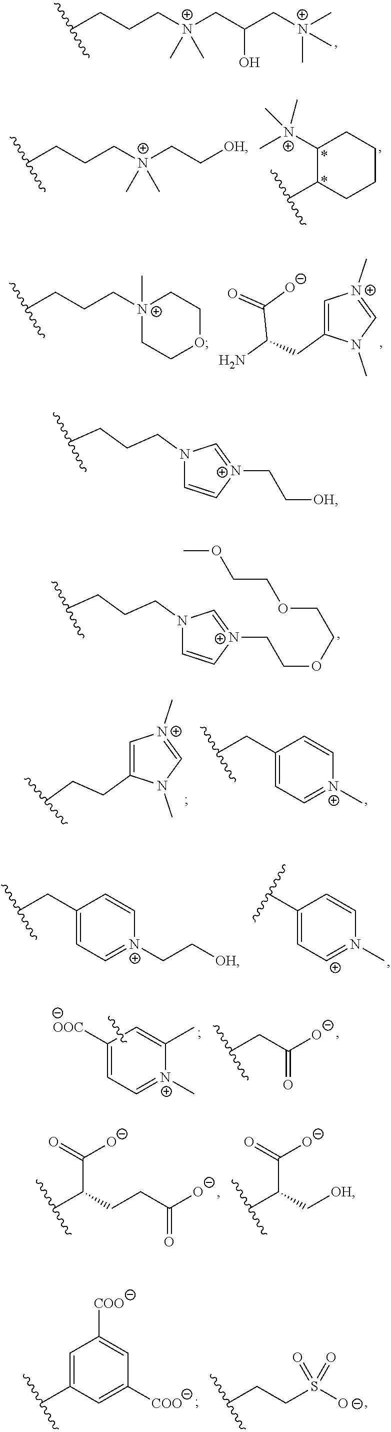

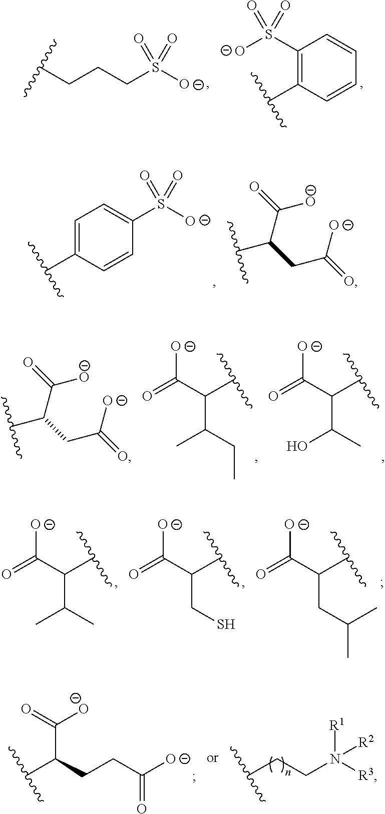

wherein R¹, R², and R³, each, independently, is a moiety selected from the group comprising: an alkyl, a glycol oligomer, or an alkyl alcohol.

In some such embodiments, the compound of Formula (I) is symmetrical, that is T and T′ are the same moiety. However, in some other embodiments, T and T′ are different moieties and, therefore, the compound is unsymmetrical.

In many other embodiments, the compound has the structure of Formula (I) or a salt thereof, wherein:
T is -(L-G)ₙ-X;
T′ is —H, —(C₁-C₆)alkyl, or -(L-G)ₙ-X;
L is —(C₂-C₅)alkyl, optionally substituted with —OH, —OCH₃, or -halogen;
G is a moiety selected from the group comprising:

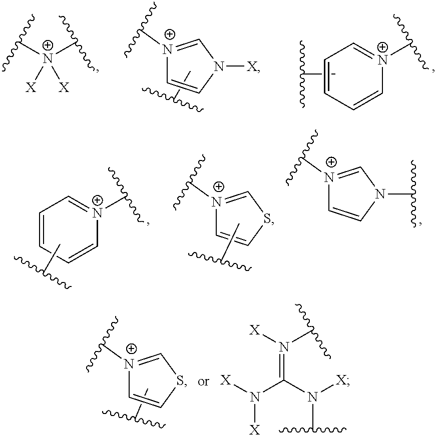

each X is, independently, a functionality selected from the group comprising: H, —(C₁-C₁₀)alkyl, —(C₂-C₆)alkenyl, —(C₂-C₆)alkynyl, and —(C₁-C₆) alkoxy, each of which is unsubstituted or substituted with one, two, or three independently selected R¹ groups; wherein

R¹ is, independently, selected from: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆)alkyl, —O(C═O)(C₁-C₆)alkyl, —O(C═O)O(C₁-C₆)alkyl, —O(C═O) OH, —O(C═O)NH₂, —O(C═O)NH(C₁-C₆)alkyl, O(C═O)N[(C₁-C₆)alkyl]₂, —NH(C═O)(C₁-C₆)alkyl, —N(C₁-C₆)alkyl(C═O)(C₁-C₆)alkyl, -halogen, —CN, —NO₂, —NH₂, —NH(C₁-C₆)alkyl, and —N[(C₁-C₆)alkyl]₂;
n is 2 to 8; and
p is 3 to 20.

Furthermore, in many embodiments, L and G are independently selected, such that, if n is 2 or more, the branched molecular tail T/T′ has multiple (n) Ls that are different and multiple (n) Gs that are different. In some embodiments, wherein n is 2, each of two L is ethyl (C₂-alkyl). In some embodiments, wherein n is 2, the first L is ethyl and the second L is propyl (C₃-alkyl). In many embodiments, L is ethyl or propyl. In addition, in some embodiments, wherein n is 2, each G is an ammonium moiety. However, in some such embodiments, wherein n is 2, the first G is an ammonium moiety and the second G is a pyridinium moiety.

In some embodiments, n is 2. In many other embodiments, n is 3. Yet, in some other embodiments, n is 4.

In many embodiments, L is unsubstituted —(C₂-C₅)alkyl. In some other embodiments, L is unsubstituted ethyl. In yet some other embodiments, L is unsubstituted propyl.

In many embodiments, G is a cationic functionality NX2:

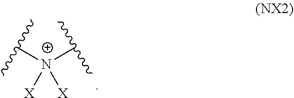

In many such embodiments, wherein G is NX2, each X is H. In some other embodiments, wherein G is NX2, X is methyl (C₁alkyl). In many embodiments, wherein G is NX2, one X is H and at least one other X is methyl. In some embodiments, wherein G is NX2, at least one X is —CH₂CH₂OH. In many embodiments, each X is independently selected.

In some embodiments, each X is, independently, H or —(C₁-C₆)alkyl. In some embodiments, at least one X is —(C₁-C₆)alkyl-OH. In some embodiments, at least one X is —CH₃CH₂OH.

In many embodiments, the compound of Formula (I) has the structure selected from the group comprising:

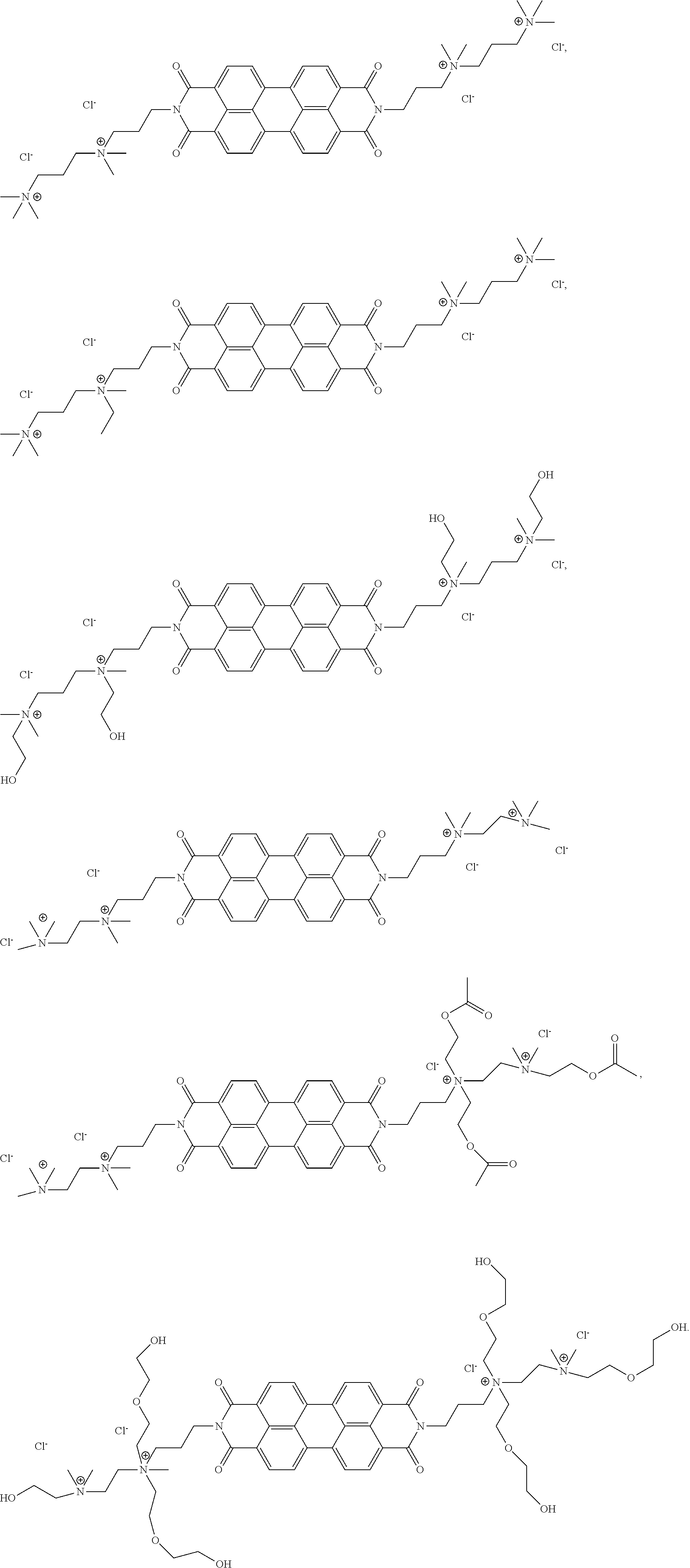

In many embodiments, wherein the molecular core is PDI, the compound has the structure of Formula (II):

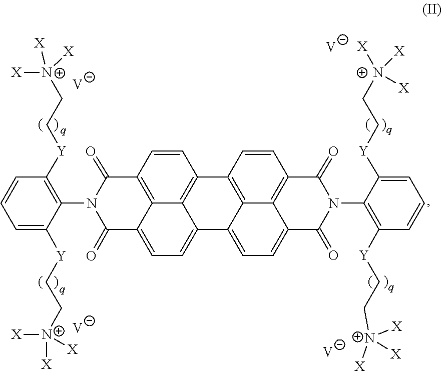

wherein
each Y is, independently, —O—, —S— or —NH—;
each q is, independently, 1 to 8; and
each X is, independently, selected from: —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups; wherein
each R¹ is, independently, selected from: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halogen, —CN, —NO₂, NH₂, NH(C₁-C₆)alkyl, and N[(C₁-C₆)alkyl]₂; and
each V is a counterion.

In some embodiments, the compound of Formula (II) has the following structure:

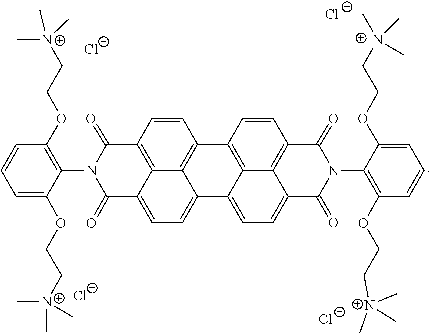 [SMILES: C[N+](C)(C)CCOc1cccc(OCC[N+](C)(C)C)c1N1C(=O)c2ccc3c4ccc5c6c(ccc(c7ccc(c2c37)C1=O)c64)C(=O)N(c1c(OCC[N+](C)(C)C)cccc1OCC[N+](C)(C)C)C5=O ; [Cl-] ; [Cl-] ; [Cl-] ; [Cl-]]

In many embodiments, the compounds, wherein the molecular core is PDI, has the structure of Formula (III):

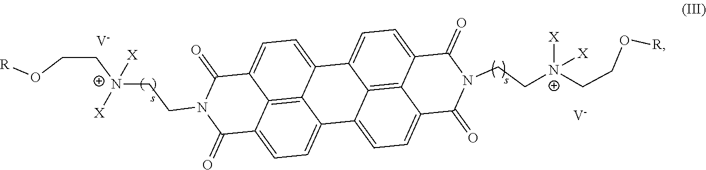

wherein
each X is, independently, selected from: —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups; wherein
each R¹ is independently selected from: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halogen, —CN, —NO₂, NH₂, NH(C₁-C₆)alkyl, and N[(C₁-C₆)alkyl]₂; and
each s is, independently, 2 to 4;
each R is, independently selected from: H, —CH₂OH, —CH₂CH₂OH, —CH₂CH₂OCH₂CH₂OH, and —CH₂CH₂OCH₂CH₂O(C₁-C₆)alkyl; and
each V is a counterion.

In some embodiments, the compound of Formula (III) has one of the following structures:

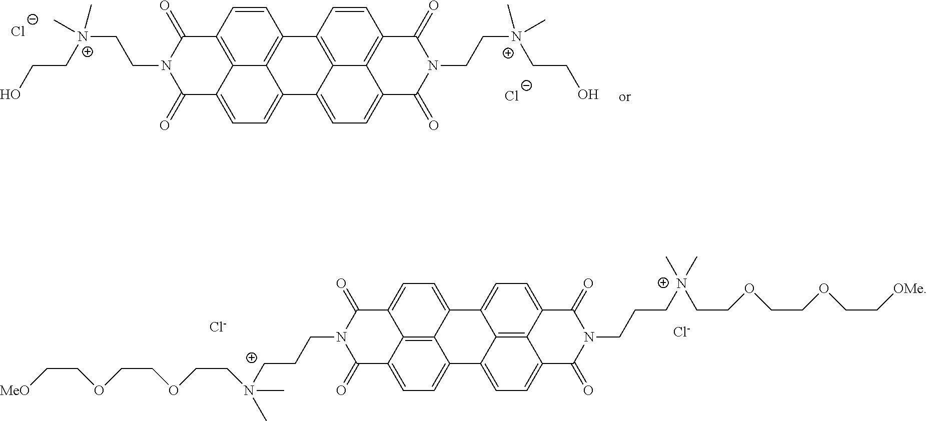

In many embodiments, the compound, wherein the molecular core is PDI, has the structure of Formula (IV):

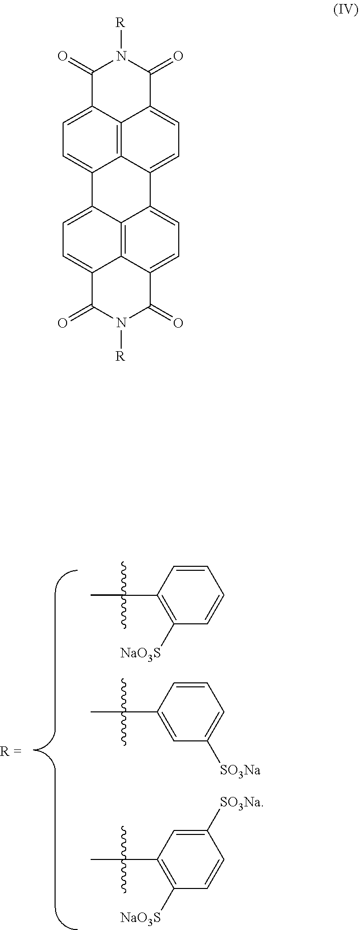

In some embodiments, the compound of Formula (IV) has the following structure:

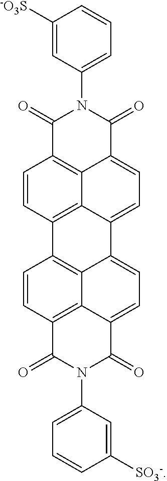

In some embodiments, the compound, wherein the molecular core is PDI, has the structure of Formula (V):

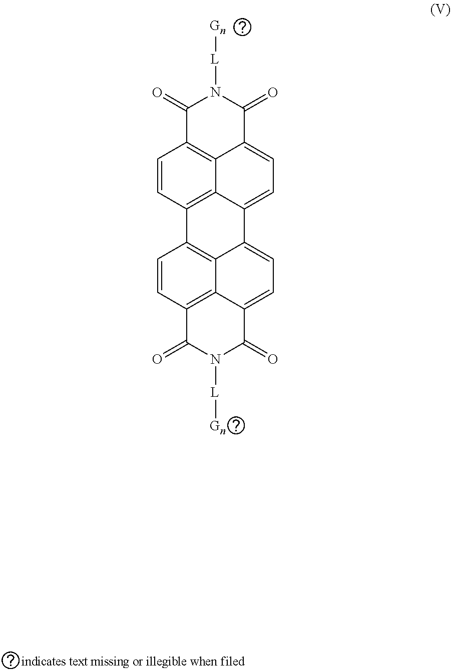

or a salt thereof, wherein
each L is, independently, selected from: —(C₁-C₆)alkyl, optionally substituted with OH, OCH₃ and halogen;
each G is, independently, selected from the group comprising:

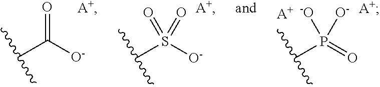

A⁺ is a cation or mixture of cations; and
n is 1 to 5.

In many embodiments, A⁺ of Formula (V).is a cation or mixture of cations selected from the group comprising, but not limited to: Li⁺, K⁺, Na⁺, NH₄⁺, and another applicable or suitable cation. In some embodiments, G of Formula (V).is

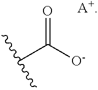

In many embodiments, L of Formula (V) is propyl. In many embodiments, n of Formula (V) is 2.

In many embodiments, the L-Gₙ functionality of Formula (V) has one or more chiral centers. In many such embodiments, the compound of Formula (V) has several stereoisomers. Accordingly, in some embodiments, the compound is present (e.g., in the electrolyte solution) as a single stereoisomer. However, in some other embodiments, the compound is present as a mixture of two or more stereoisomers in any ratio. As such, in many embodiments, the mixture comprises every possible stereoisomer of the compound, however, in some other embodiments, the mixture excludes one or more possible stereoisomers of the compound. Here, if no chirality is indicated at the stereocenter, the compound is represented by any mixture of its stereoisomers.

In many embodiments, the compound of Formula (V) has the following structure:

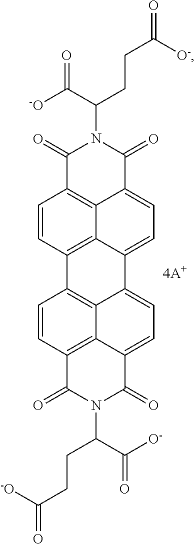

including any of its stereoisomers, and any mixture thereof:

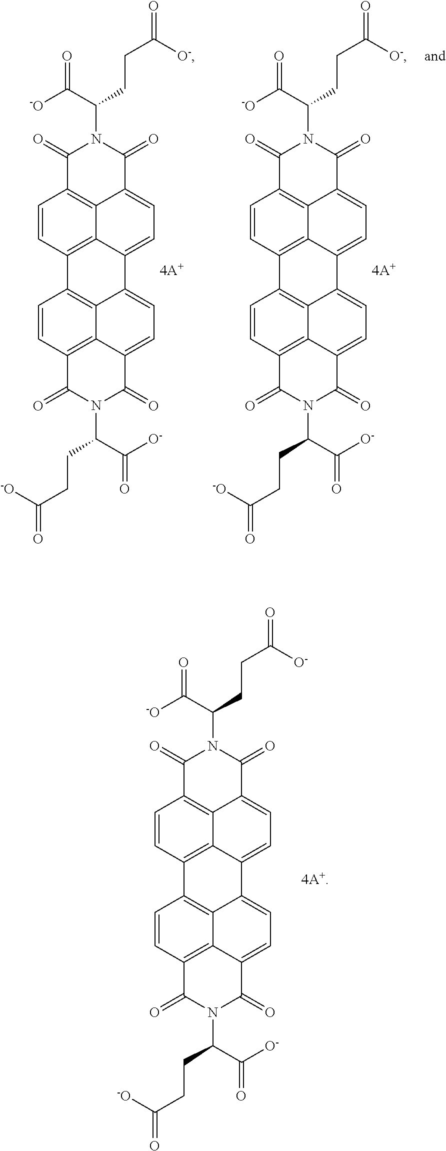

In many embodiments, the compound of Formula (V) has the following structure:

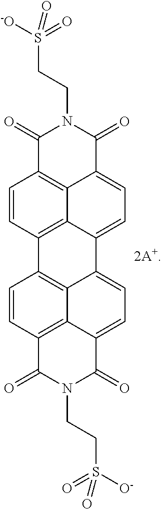

In many embodiments, wherein the molecular core is Fc, the compound has the structure selected from the group comprising:

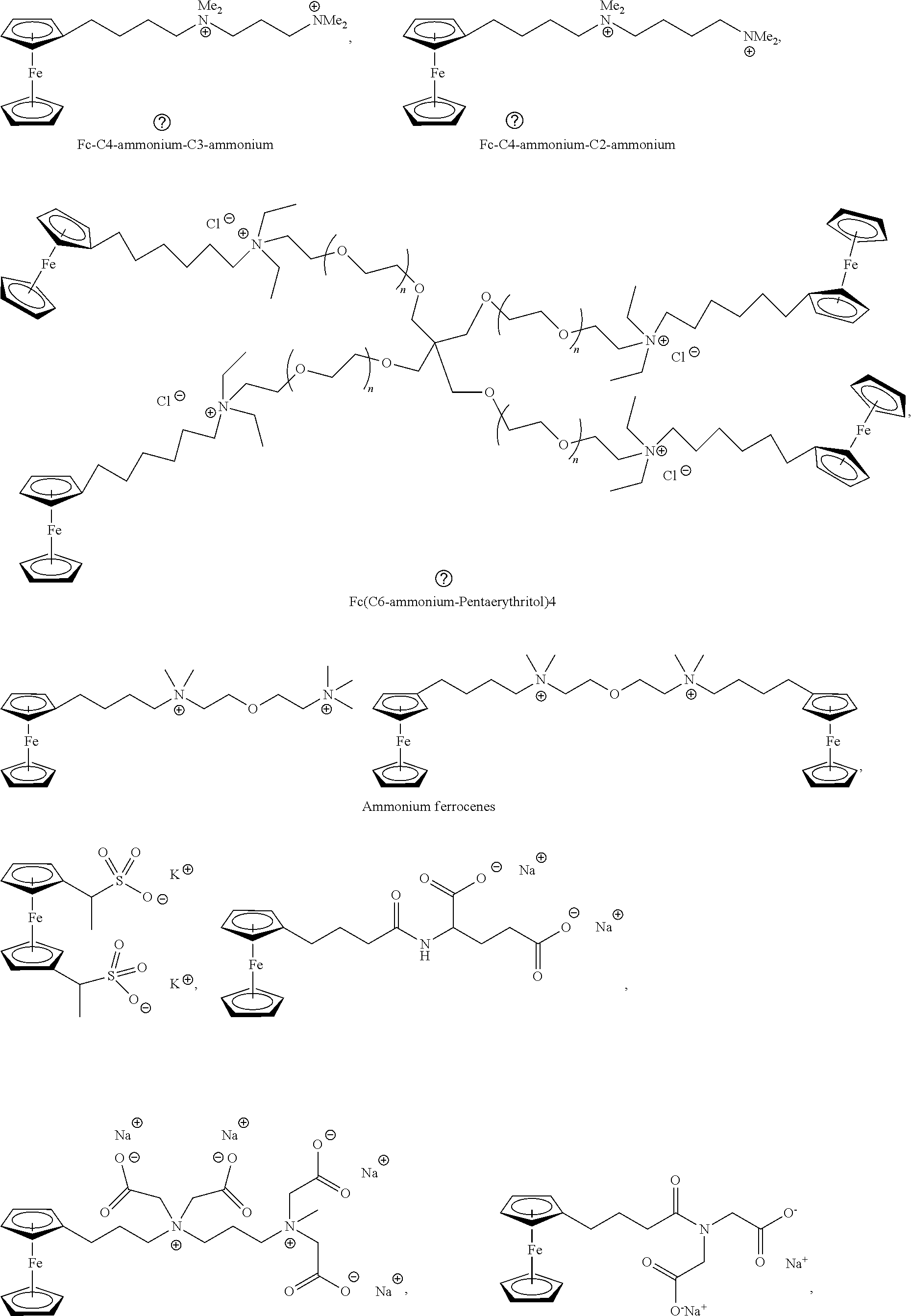

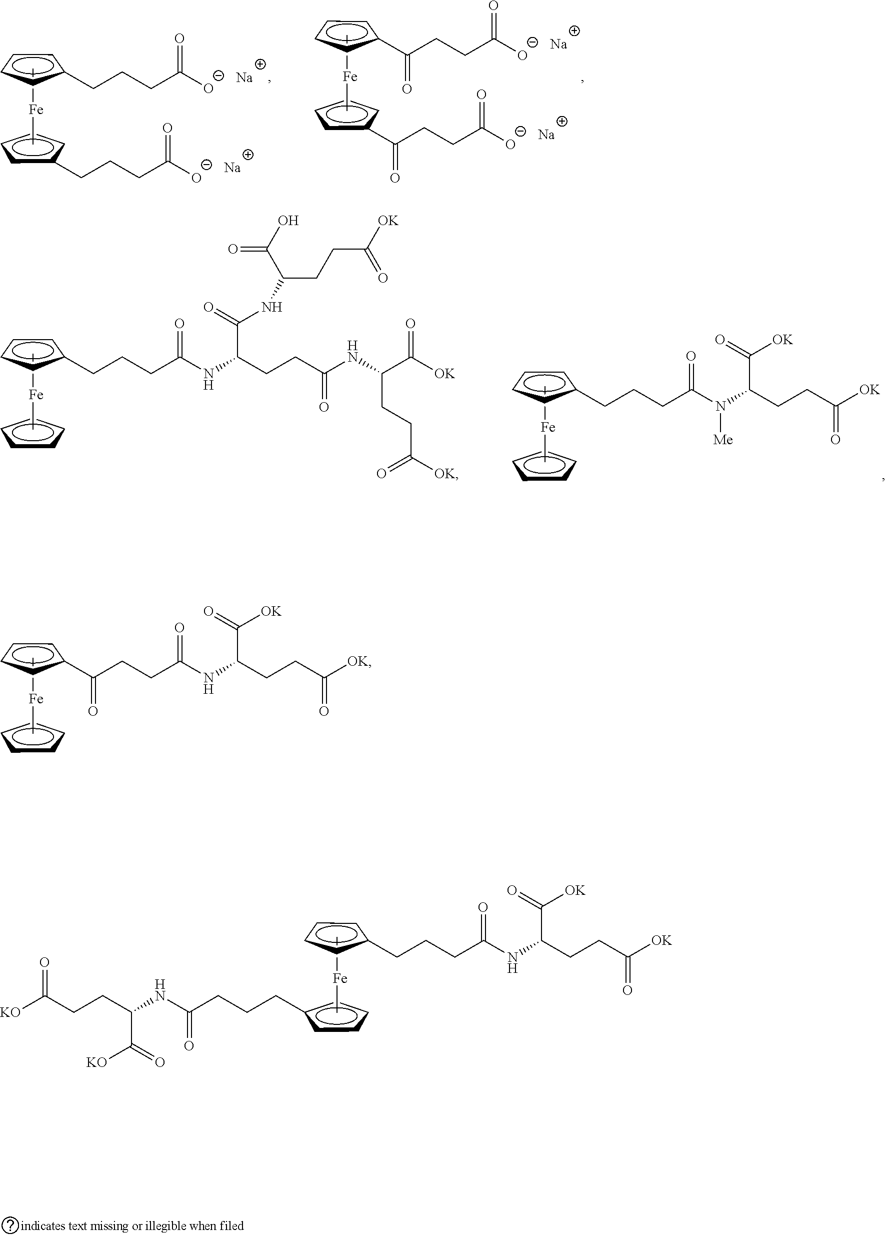

In many such embodiments, wherein the compound is Fc-C4-ammonium-C3-ammonium, Fc-C4-ammonium-C2-ammonium, Fc(C6-ammonium-Pentaerythritol)4, and ammonium ferrocenes, the positive ion on N^{⊕} is Na⁺ or K⁺.

In many embodiments, wherein the molecular core is Fc, the compound has the structure of Formula (VI):

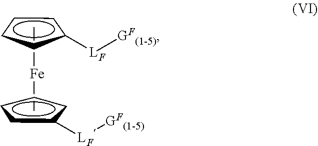

wherein
L_{F} is selected from: —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl, —(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, —(C₁-C₁₀)alkyl-aryl, each optionally substituted by one, two, or more G, —OH, —OCH₃, or -halogen;
L_{F}′ is selected from: —H, —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl, —(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, —(C₁-C₁₀)alkyl-aryl, each optionally substituted by one, two, or more G, —OH, —OCH₃, or -halogen;
each G^{F} is, independently, selected from:

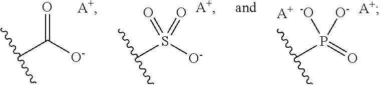

A⁺ is a cation or mixture of cations selected from the group comprising, but not limited to: Li⁺, K⁺, Na⁺, NH₄⁺, and another applicable or suitable cation; and
R² is selected from: —(C₁-C₁₀)alkyl, —(C₁-C₆)alkenyl, —(C₁-C₆)alkynyl, —(C₁-C₁₀)alkyl-aryl, -aryl, and —(C═O)—(C₁-C₆)alkyl, wherein R² is optionally substituted by one or more G.

In some embodiments, L_{F}, L_{F}′, and or G^{F}ₙ of Formula (VI) has one or more chiral centers. In many such embodiments, the compound of Formula (VI) has several stereoisomers. Accordingly, in some embodiments, the compound is present (e.g., in the electrolyte solution) as a single stereoisomer. However, in some other embodiments, the compound is present as a mixture of two or more stereoisomers in any ratio. As such, in many embodiments, the mixture comprises every possible stereoisomer of the compound, however, in some other embodiments, the mixture excludes one or more possible stereoisomers of the compound. Here, if no chirality is indicated at the stereocenter, the compound is represented by any mixture of its stereoisomers.

In many embodiments, the compound of Formula (VI) has the following structure:

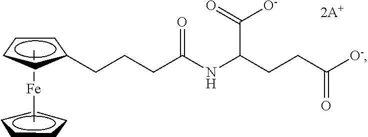

including any of its stereoisomers or any mixture thereof:

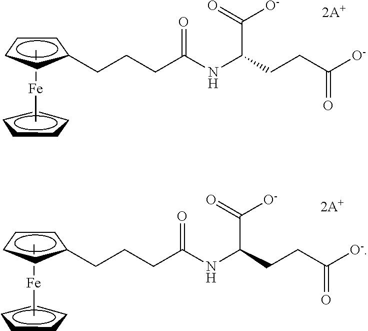

In some embodiments, the compound of Formula (VI) is:

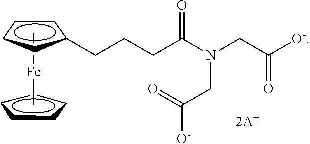

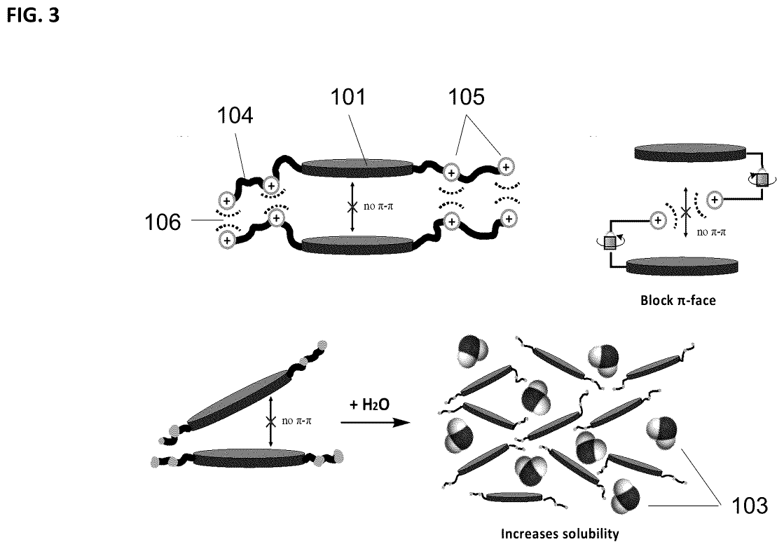

To this end, more specifically, FIG. 3 provides an illustrative example of the compound and its dissolution behavior in water, wherein the compound comprises the perylene diimide molecular core (101) enhanced with two branched molecular tails (104), wherein each branched molecular tail, in turn, comprises two ionic functionalities (105), in accordance with many embodiments. In many such embodiments, the compound comprises at least two branched molecular tails (104) attached to the redox/aromatic molecular core (101). Furthermore, in many embodiments, each branched molecular tail (104) comprises at least two ionic functionalities (105) as illustrated in FIG. 3. However, it should be noted that in some embodiments, each branched molecular tail (104) comprises at least three ionic functionalities (105), while in other embodiments, each branched molecular tail (104) comprises at least four ionic functionalities (105). In still other embodiments, each branched molecular tail (104) comprises at least five ionic functionalities (105). In some embodiments, the ionic functionalities (105) are positively charged (i.e., cationic), while in other embodiments, the ionic functionalities (105) are negatively charged (i.e., anionic), as appropriate for the requirements of a specific application.

As such, in many embodiments, and as further illustrated in FIG. 3, the electrostatic repulsion forces (106) afforded by the ionic functionalities (105) (cations in this particular example) repel the aromatic molecular cores and, as such, attenuate the TT-TT aggregation of the compound in aqueous solution. Furthermore, in many embodiments, in addition to the electrostatic repulsion forces (106), the branches of the branched molecular tails (104) further break up TT-TT stacking of the aromatic molecular cores and help create solvation shells around the ionic functionalities (105). Accordingly, in many embodiments, the branching of the branched molecular tails affords even more separation between already repelling ionic functionalities (105) on the branched molecular tails (as shown in FIG. 3, top right corner), and, thus, greatly reduces the aggregation between the molecular cores of the compound and enhances the water solubility of the compound.

In addition, in many embodiments, the repulsion forces (106) between the ionic functionalities (105) reduce entanglement of the branched molecular tails, which, otherwise, may increase viscosity of the electrolyte solution comprising the compound due to the branched molecular tails entanglement. Accordingly, in many embodiments, less entanglement between the branched molecular tails leads to lower viscosity of the compound and higher electrical conductivity.

Accordingly, in many embodiments, the branched molecular tails with charged ionic functionalities attached to the compound reduce the tendency for the aromatic organic core, such as PDI, to aggregate and, thus, improve the compound's solubility, as compared to analogous compounds functionalized with the same number and size of linear molecular tails. More specifically, in many embodiments, the incorporation of the branched molecular tails into the perylene diimide molecular core of the compound reduces π-π aggregation between PDI cores and, as such, improves water solubility of the compound.

In many embodiments, the compound is characterized by an enhanced solubility, while the electrolyte solution comprising the compound is characterized by a reduced viscosity, and an enhanced electrical conductivity in water, as compared to those of both: 1) an analogous compound with comparable solubilizing linear molecular tails comprising ionic functionalities; and 2) an analogous compound with branched molecular tails bearing no ionic functionalities. In many embodiments, the viscosity of the aqueous electrolyte solution comprising the compound is less than or equal to about 20 centipoise (cP). In many embodiments, the viscosity is or less than or equal to about 15 cP. Yet in still many embodiments, the viscosity is less than or equal to about 10 cP. Furthermore, in many embodiments, aqueous electrolyte solution comprising the compound is characterized by a conductivity of greater than or equal to about 10 mS/cm. However, in many embodiments, the conductivity is greater than or equal to about 20 mS/cm. In still many embodiments, the conductivity is greater than or equal to about 30 mS/c. Yet in some many embodiments, the conductivity is greater than or equal to about 40 mS/cm. In still some embodiments, the conductivity is greater than or equal to about 50 mS/cm. In some embodiments, the compound is characterized by an aqueous solubility of greater than or equal to about 0.275M. In some embodiments, the compound is characterized by an aqueous solubility of greater than or equal to 0.3 M.

In many embodiments, the excellent water solubility of the compound allows for preparation of aqueous electrolyte solutions of the compound without utilizing any additional solvents. In many embodiments, any water source is used to prepare the electrolyte solution comprising the compound, including, but not limited to water sources selected from the group comprising: tap water, ground water, well water, filtered water, distilled water, or deionized water, and any combination thereof, as needed and appropriate to the requirements of a specific application, with or without additional purification. However, in some embodiments, a filtration processes is carried out on the water to be used to dissolve the compound for use as the electrolyte to filter any undesirable components prior to dissolving the compound for use as the electrolyte. In some embodiments, the electrolyte solution comprising the compound further comprises supporting electrolytes, including (but not limited to) one or more salts selected from the group comprising: NaCl, KCl, NH₄Cl, Na₂SO₄, MgCl₂, and any combination or mixture thereof. In some embodiments, the electrolyte solution comprising the compound further comprises a co-solvent that increases the solubility of the compound in water. In many such embodiments, the co-solvent is a solvent selected from the group comprising: methanol, propylene carbonate, ethylene glycol, and any combination thereof.

In many embodiments, the compound is highly stable over a range of pH levels. In many embodiments, the electrolyte solution of the compound is prepared with an acidic, neutral, or basic aqueous media without any detriment to the compound's electrochemical performance. In many such embodiments, the media is neutral and the corresponding pH of the electrolyte solution comprising the compound is in a range selected from the group comprising: a neutral pH (pH of about 7), a pH of about 6 to about 8; a pH of about 6.5 to about 7.5. In other such embodiments, the media is basic and the corresponding pH of the electrolyte solution comprising the compound is in the range from about 7.5 to about 10. In still other such embodiments, the media is acidic and the corresponding pH of the electrolyte solution comprising the compound is in a range selected from the group: a pH of about 4 to about 6.5; and a pH of about 5 to about 6.5. In many embodiments, the pH of the electrolyte solution comprising the compound is neutral or close to neutral, i.e., has the pH ranging from about 5.5 to about 8.5. In many embodiments, the electrolyte solution comprising the compound is prepared using tap water, having a tap water pH, as is, without further pH adjustments.

### Redox Flow Battery

Accordingly, in many embodiments, the compound possessing such advantageous characteristics is particularly suitable for applications as an electrolyte in the redox flow battery. In many embodiments, especially wherein the molecular core is PDI, the compound serves as an anolyte. However, in some other embodiments, especially wherein the molecular core is Fc, the compound serves as a catholyte. In many embodiments, the redox flow battery employing the compound as the electrolyte is characterized by long-duration or long-lifetime, and has high energy density for energy storage applications. In some embodiments, the compound is employed as an charge storage materials in an application other than RFB. For example, in some embodiments, wherein the compound comprises the perylene diimide molecular core, the compound is reduced in an anode chamber during battery charging to store energy, and the reduced form of the compound is then oxidized during discharge to release energy. Furthermore, as another example, in some other embodiments, wherein the compound is used as a charge/energy storage material, the compound is oxidized in a cathode chamber to release electrons during battery charging to store energy, and then reduced during discharging to release the stored energy. In some embodiments, the compound comprising the PDI molecular core is employed as an anolyte and as the anode charge storage material. In some embodiments, the compound comprising the Fc molecular core is employed as a catholyte and as the cathode charge storage material.

In particular, in many embodiments, the redox flow battery comprises an electrochemical cell further comprising an anode compartment comprising an anolyte solution and a cathode compartment comprising a catholyte solution. In many embodiments, the anolyte solution comprises the compound with the PDI core decorated with the branched molecular tails comprising ionic functionalities. Furthermore, in many embodiments, the catholyte solution comprises a water soluble redox-active organic molecule, such as, for example, TEMPO, ferrocyanide, iodine, or another catholyte material. However, in some embodiments, the water soluble redox-active organic molecule is the compound with the ferrocene core decorated with the branched molecular tails comprising ionic functionalities.

In some embodiments, the electrolyte solution comprising the compound further comprises an additional supporting electrolyte or electrolytes other than the compound. In many such embodiments, the additional supporting electrolyte is (but not limited to) an inorganic or organic salt. As such, in many embodiments, the additional supporting electrolyte is an inorganic salt selected from the group comprising (but are not limited to): NaCl, KCl, LiCl, NaBr, KBr, LiBr, NaI, KI, LiI, MgCl₂, CaCl₂, MgBr₂, CaBr₂, MgI₂, and CaI₂, NH₄Cl, NH₄Br, and NH₄I. Moreover, in many embodiments, the additional supporting electrolyte is an organic salt selected from the group comprising (but are not limited to): alkylammonium chloride, alkylammonium bromide, alkylammonium iodide, sodium tosylate, and sodium besylate. In some embodiments, the electrolyte solution comprising the compound further comprises one or more co-solvents, including (but not limited to) sulfolane and propylene carbonate.

In many embodiments, the redox flow battery comprises a unit cell, which, in turn, comprises two half-cells connected by a membrane for ion transport. In many embodiments, one of the two half-cells is a cathode half-cell comprising a cathode, and the other is an anode half-cell comprising an anode. To this end, in many embodiments, a catholyte (i.e., the cathode's electrolyte) is pumped into the cathode half-cell, and an anolyte (i.e., the anode electrolyte) is pumped into the anode half-cell. Furthermore, in many embodiments, the half-cells of the redox flow battery comprise various components, including (but not limited to): electrodes, gaskets, flow plates, bipolar plates, and membranes. In many embodiments, any or all of the components of the half-cells are fabricated from non-fluorinated polymers that are not designated as highly chemically resistant. In many embodiments, the redox flow battery comprises at least one unit cell. However, in some other embodiments, the redox flow battery comprises at least two unit cells. In many embodiments, the redox flow battery comprises at least five unit cells. In many embodiments, the redox flow battery comprises at least 10 unit cells. In many embodiments, the redox flow battery comprises at least 15 unit cells. In many embodiments, the redox flow battery comprises at least 20 unit cells. In many embodiments, the redox flow battery comprises at least 25 unit cells. In many embodiments, the redox flow battery comprises at least 30 unit cells. In many embodiments, the redox flow battery comprises at least 50 unit cells. In many embodiments, the redox flow battery comprises at least 100 unit cells. In many embodiments, the redox flow battery comprises at least 150 unit cells.

The flow frame of a redox flow battery is the reaction vessel where charging and discharging takes place. The flow frame contains the porous electrode and is exposed to the flowing active electrolyte. Accordingly. In many embodiments, the reaction vessel/flow frame defining the half-cell interiors of the redox flow battery is made of at least one non-fluorinated polymer. In many embodiments, the non-fluorinated polymer used to manufacture the flow frame is polymer selected from the group (but are not limited to) comprising: polyethylene, polypropylene, PMP, polybutene-1, PVC, polystyrene, PMMA, ABS, nylon, POM, polycarbonate, nylon, PEEK, and any combination thereof. In some embodiments, the reaction vessel is made of copolymers derived from two or more of the aforementioned polymers. In many embodiments, the flow frame is made of titanium. In several embodiments, the flow frame is made of stainless steel.

In many embodiments, the redox flow battery includes a first half-cell comprising the anolyte solution and a second half-cell comprising the catholyte solution, wherein the anolyte solution comprises the compound with the PDI molecular core, and wherein one or more of the redox flow battery's components (e.g., an electrode, gasket, flow frame, bipolar plate, membrane) is made from a material that is not designated as highly chemically resistant. In many embodiments, the redox flow battery is constructed from at least one hard material for the flow frame, hard plumbing connections and electrolyte tanks, at least one soft material for the seals and soft tubing, a conductive hard material for the flow field, a conductive porous electrode, and a membrane. In some embodiments, the redox flow battery's components are made from non-fluorinated polymers. In several embodiments, the non-fluorinated polymer is a polyolefin, a polyether, polyketone, a polyamide, a polyurea, a natural rubber, or a combination thereof.

In many embodiments, the solution comprising the compound as the electrolyte is compatible with various materials, including materials that are not necessarily designated as highly chemically resistant (see, e.g., U.S. Provisional Patent Application No. 63/488,373 filed Mar. 3, 2023, the disclosure of which is incorporated herein by reference). Here, the term “compatible” is defined as having minimal or no deleterious electrochemical or physical interactions between the aqueous solution of the compound and any component of the electrochemical cell that contains such solution, that would preclude long-duration and multi-year lifetime of the cell.

Many embodiments provide that the redox flow battery (and, therefore, the compound) are compatible with functional groups such as small molecules including (but not limited to) sodium besylate, sodium tosylate, propylene carbonate, sulfolane, and any combinations thereof.

In many embodiments, the redox flow battery is characterized by a capacity retention greater than or equal to about 90% over a period of at least two weeks. In many embodiments, the capacity retention is greater than or equal to about 95% over a period of at least two weeks. In still many embodiments, the capacity retention is greater than or equal to about 99% over a period of at least two weeks. In many such embodiments, the capacity retention is greater than or equal to about 99.9% over a period of at least two weeks.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., s or sec, second(s); min, minute(s); h or hr, hour(s); and the like.

### Example 1—Synthesis of Compound 1

N-(4-ferrocenylbutanoyl)-L-glutamate (8.36 g, 20.8 mmol, 1.0 equiv), 1-(bis(dimethylamino)methylene)-1H-benzo[d][1,2,3]triazole-1-ium 3-oxide hexafluorophosphate (17.4 g, 45.8 mmol, 2.2 equiv), and N-ethyl-N-isopropylpropan-2-amine (5.92 g, 45.8 mmol, 2.2 equiv) were dissolved in THF (75 mL) in a 250 mL round-bottom flask. After stirring for 5 min, a solution of dimethyl L-glutamate hydrochloride (8.03 g, 45.8 mmol, 2.2 equiv) in THF (25 mL) was added dropwise. The mixture was stirred at room temperature and monitored by HPLC, showing complete conversion after 36 h. The reaction was quenched with saturated sodium bicarbonate (100 mL) and extracted with diethyl ether (3×100 mL). The combined organic layers were washed with 2 M HCl (100 mL) and brine (100 mL), dried over anhydrous sodium sulfate, and concentrated to yield 8.55 g of brown oil.

The brown oil was dissolved in THF (50 mL) and cooled to 0° C., where lithium hydroxide (1.17 g, 49.0 mmol, 4.1 equiv) in water (75 mL, 0.54 M) was added. After stirring for 2 h, the reaction was neutralized with 3 N HCl and THF was removed. The aqueous layer was purged with nitrogen and adjusted to pH 3 with 2 N HCl. The aqueous solution was extracted with EtOAc (100 mL, followed by 50 mL), washed with 2 M HCl (2×100 mL) and brine (100 mL), then concentrated to a brown oil, yielding 6.84 g (87%). The crude product was dissolved in THF, passed through silica, and precipitated with heptane. After drying, Compound 1 was isolated as a yellow solid (4.36 g):

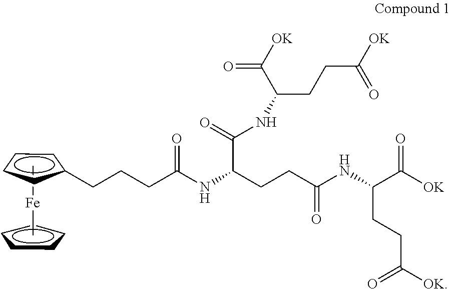

### Example 2—Synthesis of Compound 2

A solution of 4-ferrocene butyric acid (1.80 g, 6.61 mmol) and (2.01 g, 7.28 mmol) in THF (33.1 mL) at 23° C. was treated with 4-methylmorpholine (736 mg, 7.28 mmol) and stirred for 5 min. Dimethyl methyl-L-glutamate (1.49 g, 7.87 mmol) was added, and the mixture was stirred at RT for 48 h. The reaction mixture was poured into water, extracted with EtOAc, and washed with saturated sodium bicarbonate. The solution was concentrated to an oil, which was dried under vacuum overnight. The oil was then dissolved in water (25 mL) and treated with lithium hydroxide (153 mg, 6.39 mmol) for 2 h at room temperature. The reaction was quenched with 3 N HCl to pH 7, and THF was removed by rotary evaporation, yielding Compound 2 as a dark yellow oil:

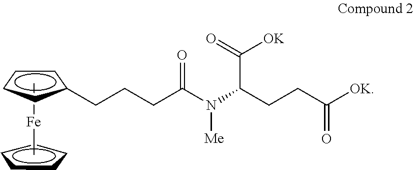

### Example 3—Solubility Data

4-ferrocene butyric acid was dissolved to a maximum concentration of 0.251M, after which solid precipitated from the solution.

N-(4-ferrocenylbutanoyl)-L-glutamate was dissolved to a concentration of 0.67M with a viscosity of 3.7 cP.

### Doctrine of Equivalents

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A compound for use as an electrolyte in a redox flow battery comprising:
a molecular core, wherein the molecular core is a redox moiety, covalently functionalized with
one or more branched molecular tails, wherein the one or more branched molecular tails, together, further comprise
at least two ionic functionalities.

2. The compound of claim 1, wherein the molecular core is perylene diimide, or ferrocene.

3. The compound of claim 1, wherein the molecular core is perylene diimide and the compound has a Formula (I): 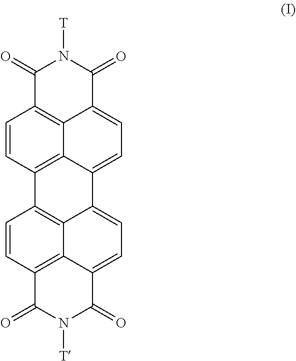
or a salt thereof, wherein:
T and T′ are, independently selected from the group consisting of: 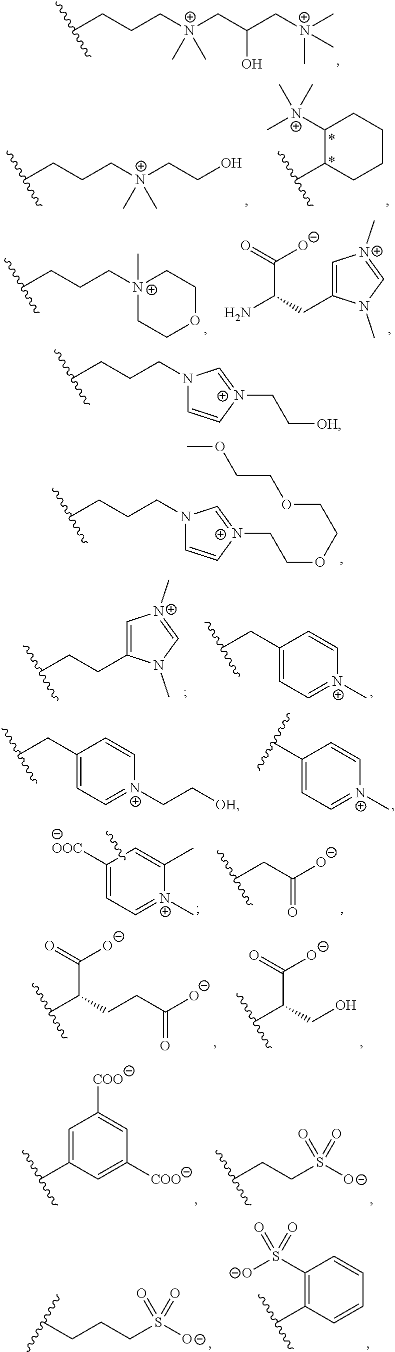 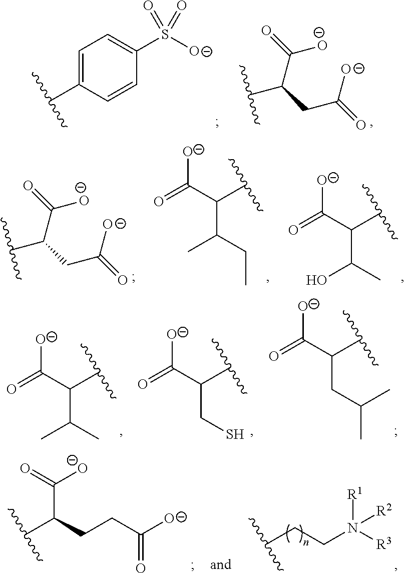
wherein R¹, R², R³ is, independently, an alkyl, a glycol oligomer, or an alkyl alcohol.

4. The compound of claim 1, wherein the molecular core is perylene diimide and the compound has a Formula (I): 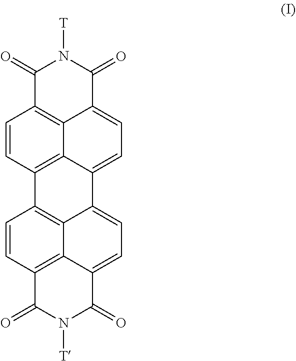
or a salt thereof, wherein:
T is -(L-G)ₙ-X;
T′ is —H, —(C₁-C₆)alkyl, or -(L-G)ₙ-X;
L is —(C₂-C₅)alkyl, optionally substituted with —OH, —OCH₃, -halogen;
G is a moiety selected from the group consisting of: 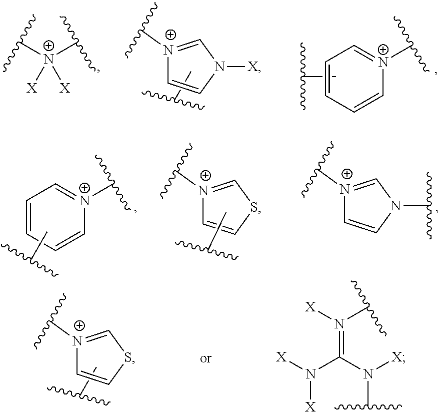
each X is, independently, —H, —(C₁-C₁₀)alkyl, —(C₂-C₆)alkenyl, —(C₂-C₆)alkynyl, and —(C₁-C₆) alkoxy, each of which is unsubstituted or substituted with 1, 2, or 3 independently selected R¹ groups;
each R¹ is independently —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂;
n=2 to 8; and
p=3 to 20.

5. The compound of claim 4, wherein T and T′ are each, independently, -(L-G)ₙ-X.

6. The compound of claim 5, wherein T and T′ are the same.

7. The compound of claim 5, wherein T and T′ are different.

8. The compound of claim 4, wherein L is unsubstituted —(C₂-C₅)alkyl.

9. The compound of claim 4, wherein L is ethyl or propyl.

10. The compound of claim 4, wherein n is 2, 3, or 4.

11. The compound of claim 4, wherein G is

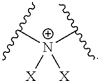

12. The compound of claim 11, wherein X is —H, -methyl or —CH₂CH₂OH.

13. The compound of claim 11, wherein each X is, independently, —H or —(C₁-C₆)alkyl.

14. The compound of claim 11, wherein at least one X is —CH₃CH₂OH.

15. The compound of claim 4, wherein the compound of Formula (I) is a compound selected from the group consisting of:

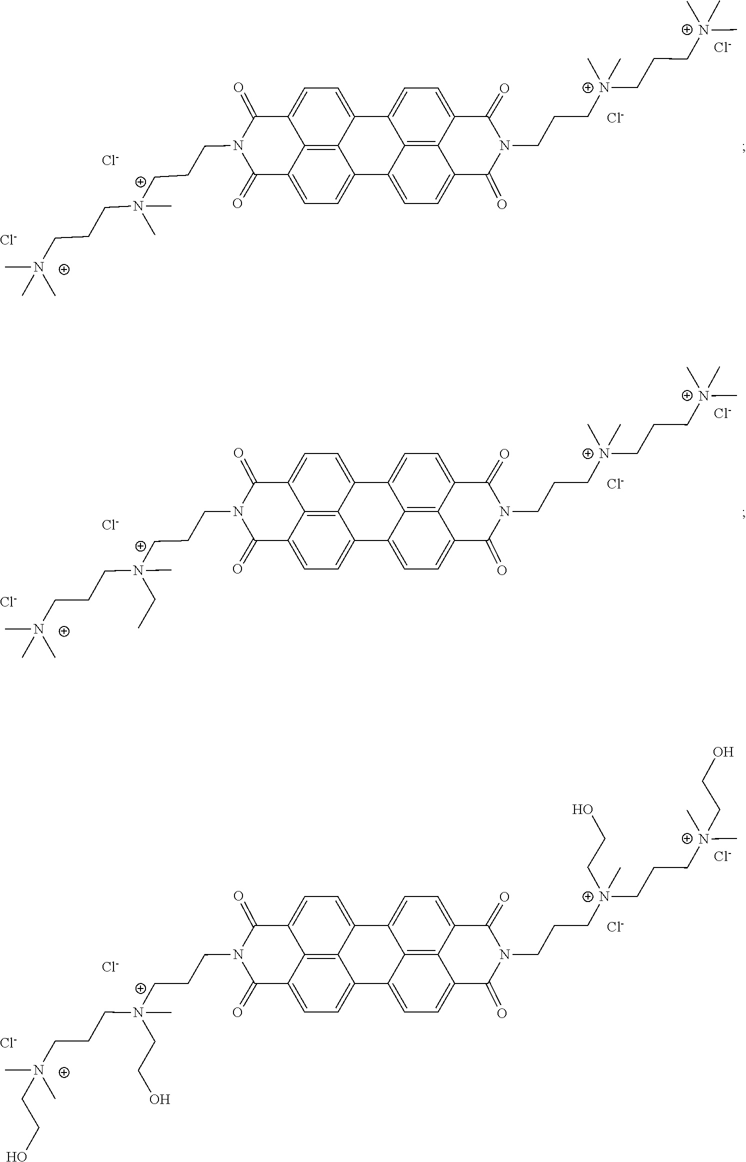

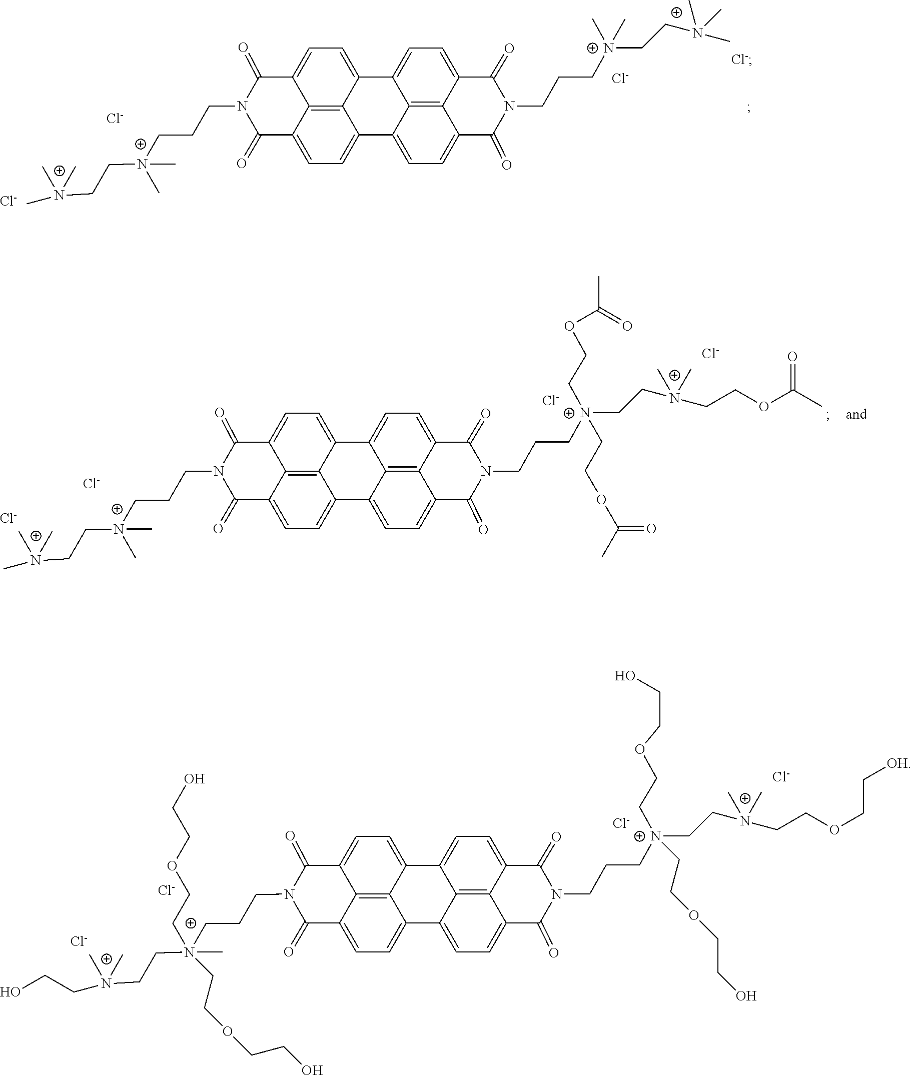

16. The compound of claim 1, wherein the molecular core is perylene diimide and the compound has a Formula (II): 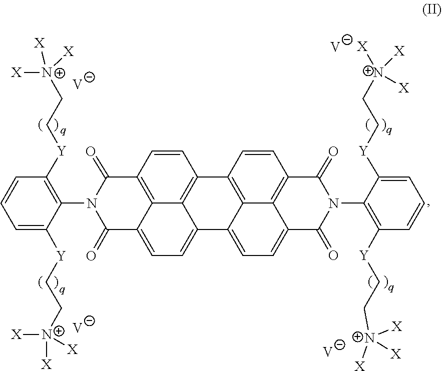
wherein
each Y is, independently, —O—, —S— or —NH—;
each q is, independently, 1 to 8; and
each X is, independently, selected from the group consisting of: —H, —(C₁-C₁₀)alkyl, —(C₂-C₆)alkenyl, —(C₂-C₆)alkynyl, and —(C₁-C₆)alkoxy, each of which is unsubstituted or substituted with 1, 2, or 3 independently selected R¹ groups;
each R¹ is, independently, selected from the group consisting of: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆), —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂; and
each V is a counterion.

17. The compound of claim 16, wherein the compound of Formula (II) is:

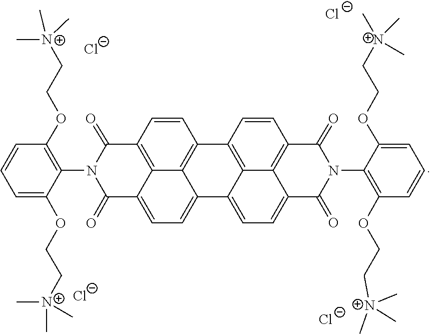 [SMILES: C[N+](C)(C)CCOc1cccc(OCC[N+](C)(C)C)c1N1C(=O)c2ccc3c4ccc5c6c(ccc(c7ccc(c2c37)C1=O)c64)C(=O)N(c1c(OCC[N+](C)(C)C)cccc1OCC[N+](C)(C)C)C5=O ; [Cl-] ; [Cl-] ; [Cl-] ; [Cl-]]

18. The compound of claim 1, wherein the molecular core is perylene diimide and the compound has a Formula (III): 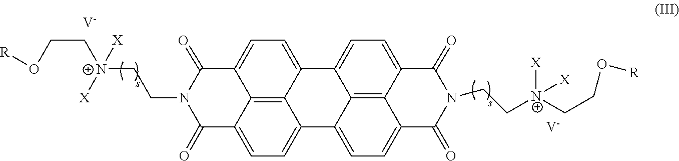
wherein:
each X is, independently, selected from the group consisting of: —H, —(C₁-C₁₀)alkyl, —(C₂-C₆)alkenyl, —(C₂-C₆)alkynyl, and —(C₁-C₆)alkoxy, each of which is unsubstituted or substituted with 1, 2, or 3 independently selected R¹ groups;
each R¹ is, independently, selected from the group consisting of: —OH, —O(C₁-C₆)alkyl, —O(C₁-C₆)alkyl-O(C₁-C₆)alkyl, —O(C₁-C₆)alkyl-O(C₁-C₆)alkyl-O(C₁-C₆)alkyl, —[O(C₁-C₆)alkyl]ₚ-O(C₁-C₆)alkyl, —O(C═O)(C₁-C₆)alkyl, —O(C═O)O(C₁-C₆)alkyl, —O(C═O)OH, —O(C═O)NH₂, —O(C═O)NH(C₁-C₆)alkyl, O(C═O)N[(C₁-C₆)alkyl]₂, —NH(C═O)(C₁-C₆)alkyl, N(C₁-C₆)alkyl(C═O)(C₁-C₆)alkyl, halo, —CN, —NO₂, NH₂, NH(C₁-C₆)alkyl, and N[(C₁-C₆)alkyl]₂;
each s is, independently, 2 to 4;
each R is, independently, —H, —CH₂OH, —CH₂CH₂OH, —CH₂CH₂OCH₂CH₂OH, or —CH₂CH₂OCH₂CH₂O(C₁-6)alkyl; and
each V⁻ is a counterion.

19. The compound of claim 18, wherein the compound of Formula (III) is:

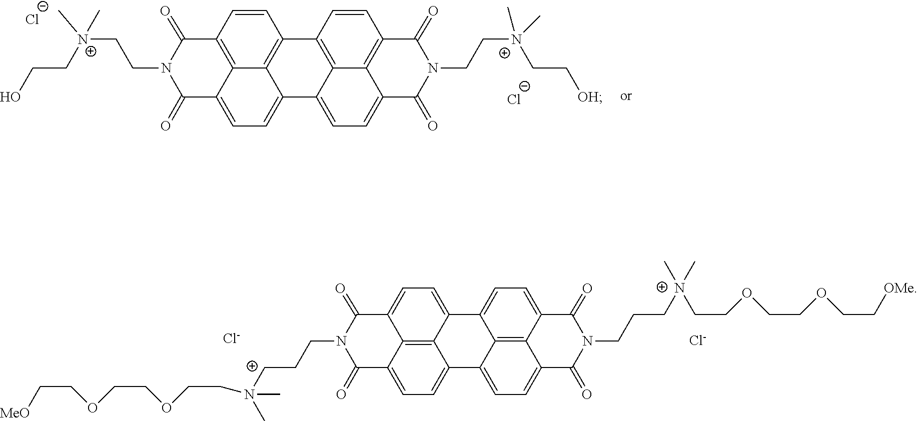

20. The compound of claim 1, wherein the molecular core is perylene diimine and the compound has a Formula (IV): 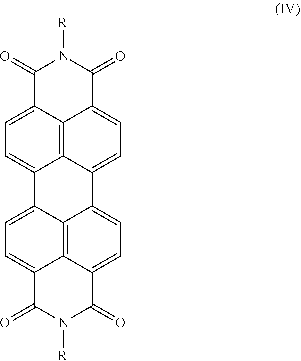
wherein
R is

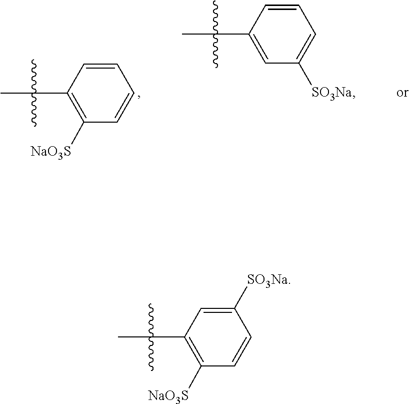

21. The compound of claim 20, wherein the compound of Formula (IV) is:

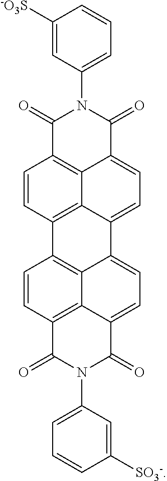

22. The compound of claim 1, wherein the molecular core is perylene diimide and the compound has a Formula (V): 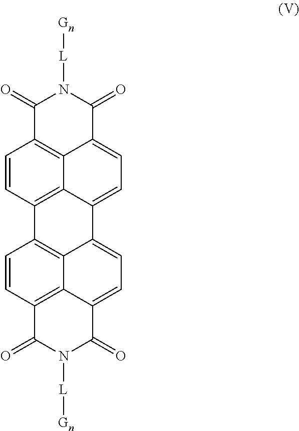
or a salt thereof, wherein
L is —(C₁-C₆)alkyl;
each G is 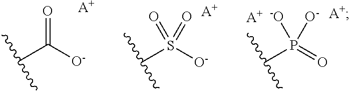
A⁺ is one or more cations; and
n=1 to 5.

23. The compound of claim 22, wherein L is substituted with —OH, —OCH₃, or -halogen.

24. The compound of claim 22, wherein each A⁺ is one or more cations selected from the group consisting of: Li⁺, K⁺, Na⁺, NH₄⁺, and another suitable cation.

25. The compound of claim 22, wherein each G is

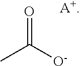

26. The compound of claim 22, wherein each L is propyl.

27. The compound of claim 22, wherein n is 2.

28. The compound of claim 22, wherein L-Gₙ of Formula (V) has at least one chiral center.

29. The compound of claim 28, wherein the formula (V) has at least one stereoisomer.

30. The compound of claim 22, wherein the compound of Formula (V) is: 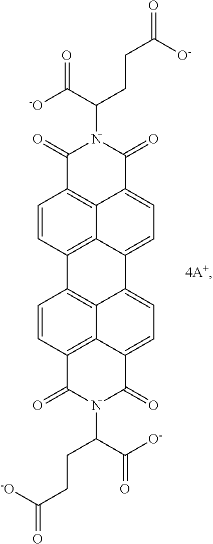
and any one or any combination of its stereoisomers:

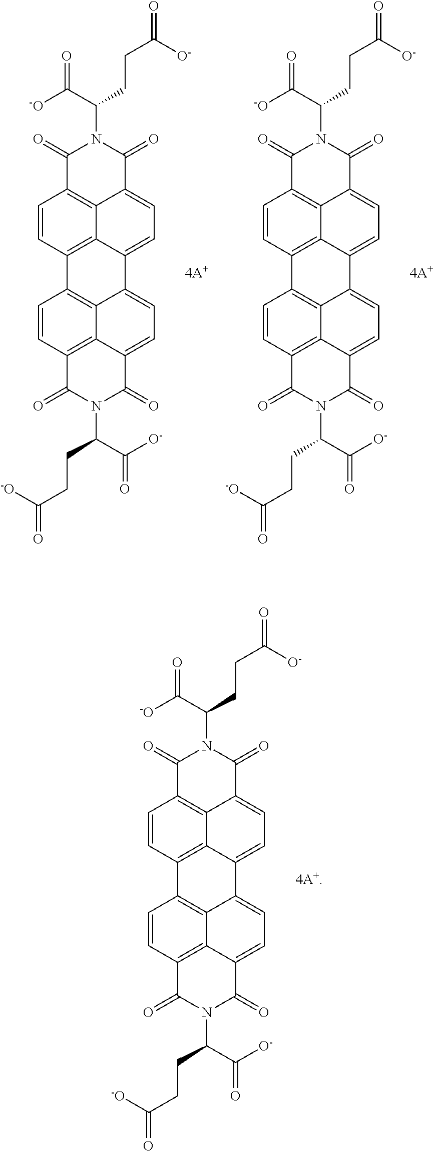

31. The compound of claim 30, wherein A⁺ is one or more cations selected from the group consisting of: Li⁺, K⁺, Na⁺, NH₄⁺, and another applicable.

32. The compound of claim 22, wherein the compound of Formula (V) is:

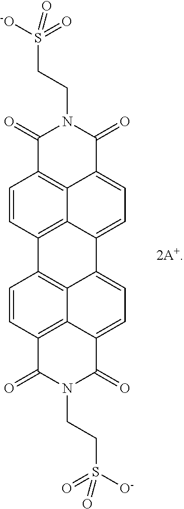

33. The compound of claim 1, wherein the molecular core is ferrocene and the compound has a structure selected from the group consisting of:

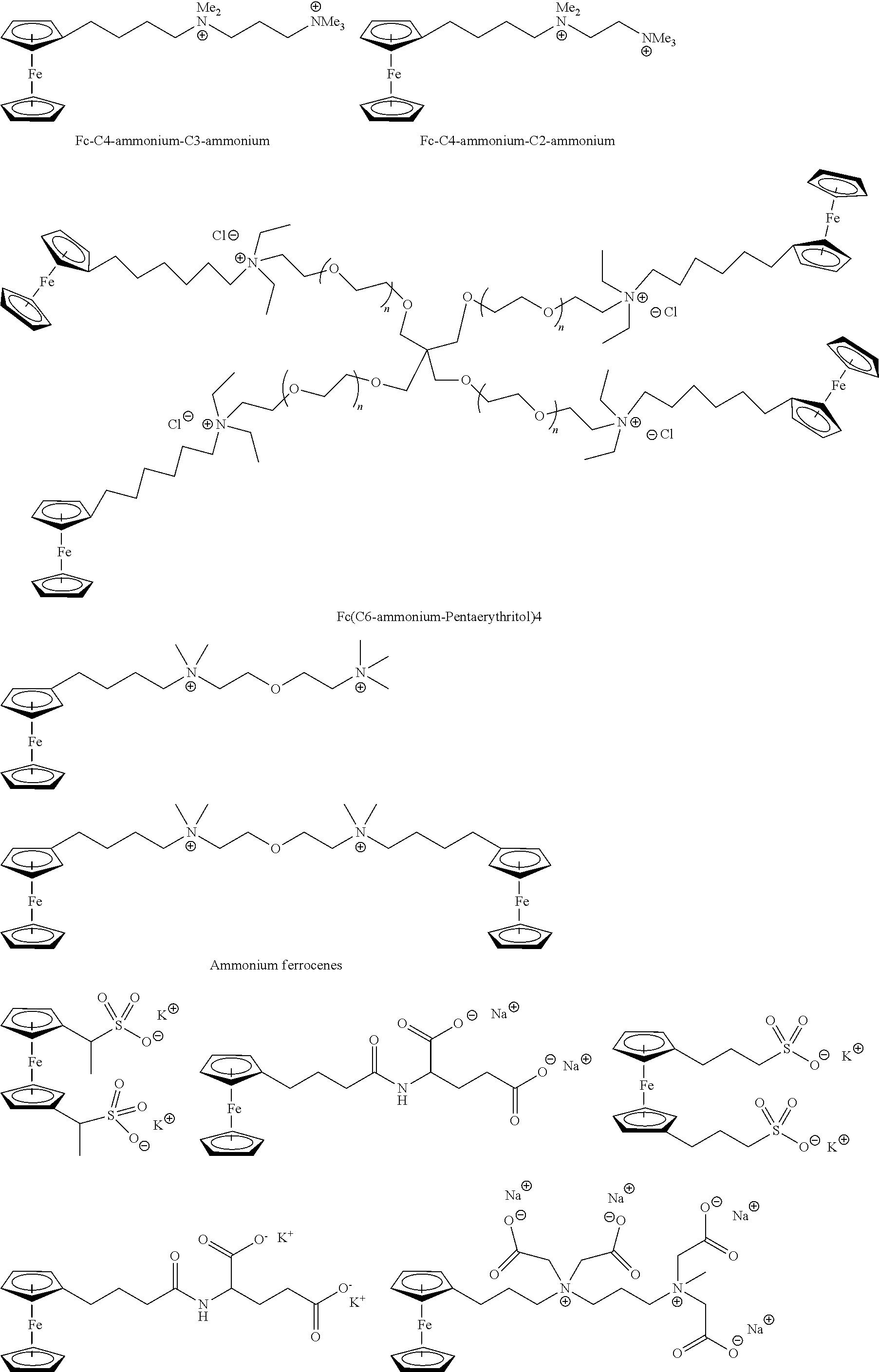

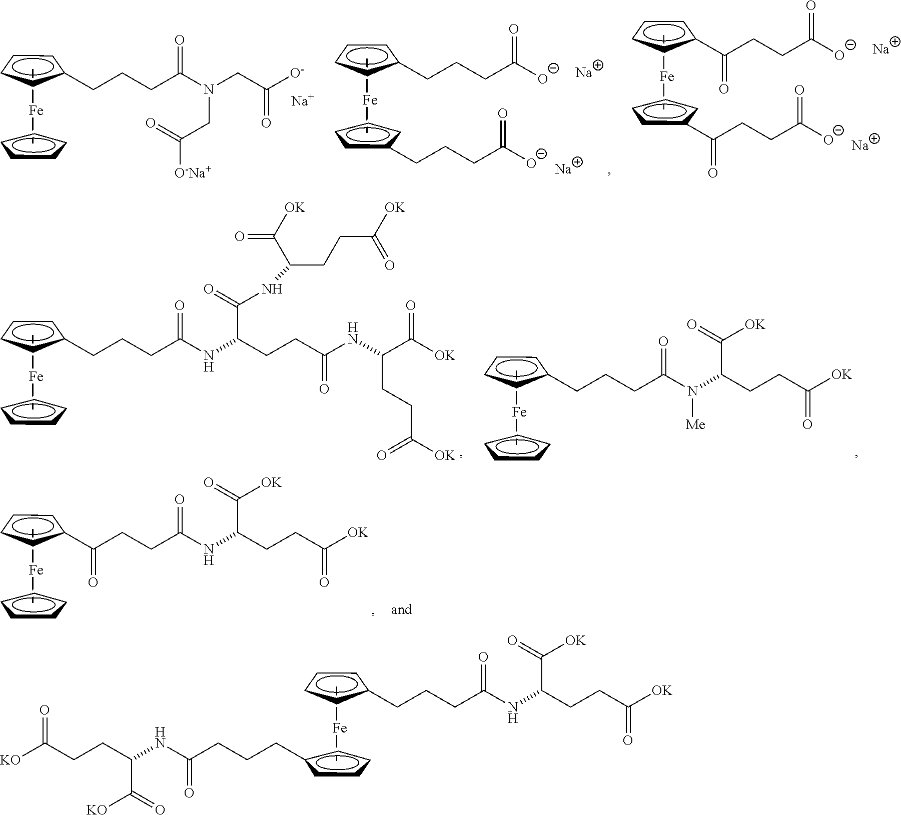

34. The compound of claim 1, wherein the molecular core is ferrocene and the compound has a Formula VI: 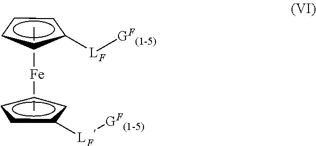
wherein:
L_{F} is selected from the group consisting of: —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
L_{F}′ is selected from the group consisting of: —H, —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
G^{F} is selected from the group consisting of: 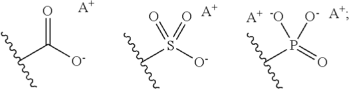
G^{F} is greater than or equal to 2;
A⁺ is one or more cations and
R² is —(C₁-C₁₀)alkyl, —(C₁-C₆)alkenyl, —(C₁-C₆)alkynyl, —(C₁-C₁₀)alkyl-aryl, -aryl, or —(C═O)—(C₁-C₆)alkyl.

35. The compound of claim 34, wherein L^{F} is substituted by at least one group selected from the group consisting of: G^{F}, —OH, —OCH₃, and -halogen.

36. The compound of claim 34, wherein L^{F}′ is substituted at least one group selected from the group consisting of: G^{F}, —OH, —OCH₃, and -halogen.

37. The compound of claim 34, wherein R² is substituted by at least one G^{F}.

38. The compound of claim 34, wherein A⁺ is one or more cations selected from the group consisting of: Li⁺, K⁺, Na⁺, NH₄⁺, and another suitable cation.

39. The compound of claim 34, wherein L_{F}, L_{F}′, and or G^{F}ₙ has at least one chiral center.

40. The compound of claim 34, wherein the compound has at least one stereoisomer.

41. The compound of claim 34, wherein the compound of Formula (VI) is 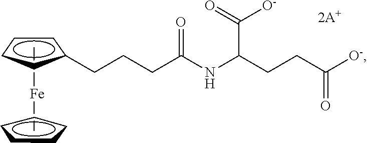
and any one or any combination of its stereoisomers:

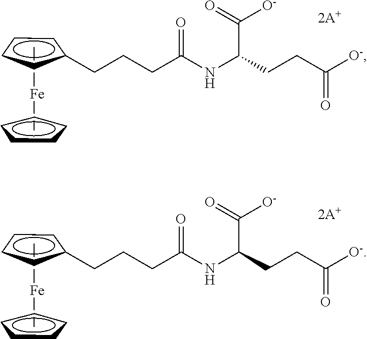

42. The compound of claim 34, wherein the compound of Formula (VI) is:

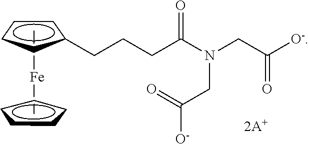

43. A redox flow battery comprising:
a first half-cell comprising a first electrode and a first solution of a first electrolyte, wherein the first electrolyte is the compound with a perylene diimide molecular core of with a perylene diimide molecular core according to Formula (I): 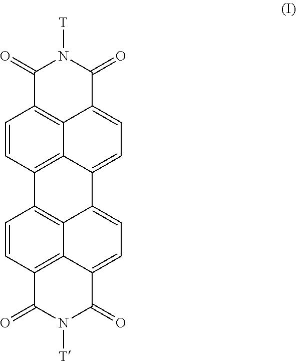
or a salt thereof, wherein:
T and T′ are, independently selected from the group consisting of: 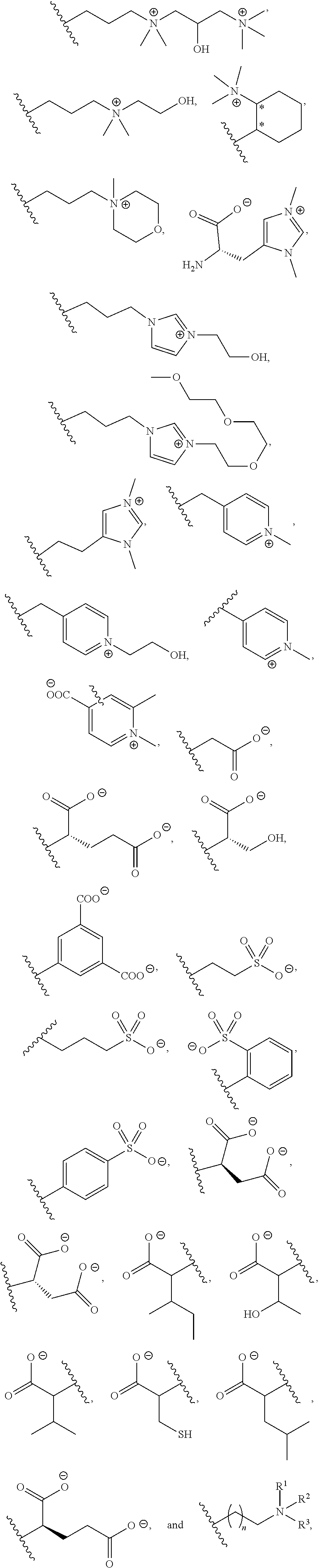
wherein R¹, R², R³ is, independently, an alkyl, a glycol oligomer, or an alkyl alcohol;
a second half-cell comprising a second electrode and a second solution of a second electrolyte; and
a separator interposed between the first half-cell and the second half cell.

44. The redox flow battery of claim 42, wherein the second electrolyte is the compound with a ferrocene molecular core and the compound has a Formula VI: 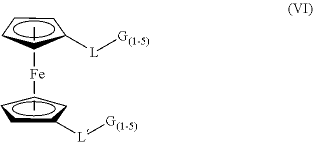
wherein:
L_{F} is selected from the group consisting of: —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
L_{F}′ is selected from the group consisting of: —H, —(C₁-C₁₀)alkyl, —(C₁-C₆)alkenyl, —(C₁-C₆)alkynyl, —(C₁-C₆)alkyl-O(C₁-C₆)alkyl, —(C₁-C₆)alkyl-O—(C═O)—(C₁-C₆)alkyl, —(C₁-C₆)alkyl-(C═O)—O—(C₁-C₆)alkyl, —(C₁-C₆)alkyl-NH—(C═O)(C₁-C₆)alkyl, —(C₁-C₆)alkyl-NR²—(C═O)(C₁-C₆)alkyl, —(C₁-C₆)alkyl-(C═O)—NH—(C₁-C₆)alkyl, —(C₁-C₆)alkyl-(C═O)—NR²—(C₁-C₆)alkyl, or —(C₁-C₁₀)alkyl-aryl;
G^{F} is selected from the group consisting of: 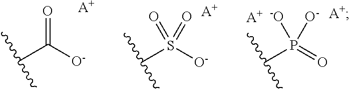
G^{F} is greater than or equal to 2;
A⁺ is one or more cations and
R² is —(C₁-C₁₀)alkyl, —(C₁-C₆)alkenyl, —(C₁-C₆)alkynyl, —(C₁-C₁₀)alkyl-aryl, -aryl, or —(C═O)—(C₁-C₆)alkyl.
